(19) United States
(12) Patent Application Publication
(10) Pub. No.: US 2022/0285027 A1
(43) Pub. Date: Sep. 8, 2022

(54) PREDICTION OF DISEASE STATUS

(71) Applicant: Hoffmann-La Roche Inc., Little Falls, NJ (US)

(72) Inventors: Christian GOSSENS, Basel (CH); Florian LIPSMEIER, Basel (CH); Cedric Andre Marie Vincent Geoffrey SIMILLION, Lutzelfluh-Goldbach (CH); Michael LINDEMANN, Schopfheim (DE)

(73) Assignee: Hoffmann-La Roche Inc., Little Falls, NJ (US)

(21) Appl. No.: 17/705,726

(22) Filed: Mar. 28, 2022

Related U.S. Application Data
(63) Continuation of application No. PCT/EP2020/077207, filed on Sep. 29, 2020.

(30) Foreign Application Priority Data
Sep. 30, 2019 (EP) 19200522.1

(51) Int. Cl. G16H 50/20; G06N 20/20; G06N 20/10

(52) U.S. Cl. CPC G16H 50/20; G06N 20/20; G06N 20/10

(57) ABSTRACT

A machine learning system (110) for determining at least one analysis model for predicting at least one target variable indicative of a disease status is proposed. The machine learning system (110) comprises: at least one communication interface (114) configured for receiving input data, wherein the input data comprises a set of historical digital biomarker feature data, wherein the set of historical digital biomarker feature data comprises a plurality of measured values indicative of the disease status to be predicted; at least one model unit (116) comprising at least one machine learning model comprising at least one algorithm; at least one processing unit (112), wherein the processing unit (112) is configured for determining at least one training data set and at least one test data set from the input data set, wherein the processing unit (112) is configured for determining the analysis model by training the machine learning model with the training data set, wherein the processing unit (112) is configured for predicting the target variable on the test data set using the determined analysis model, wherein the processing unit (112) is configured for determining performance of the determined analysis model based on the predicted target variable and a true value of the target variable of the test data set.

### CROSS REFERENCE TO RELATED APPLICATIONS

This application is a continuation of International Application No. PCT/EP2020/077207, filed Sep. 29, 2020, which claims priority to EP Application No. 19200522.1, filed Sep. 30, 2019, which are incorporated herein by reference in their entireties.

### TECHNICAL FIELD

The present invention relates to the field of digital assessment of diseases. In particular, the present invention relates to a machine learning system for determining at least one analysis model for predicting at least one target variable indicative of a disease status and a computer-implemented method for determining at least one analysis model for predicting at least one target variable indicative of a disease status. Moreover, the present invention relates to a computer program and a computer-readable storage medium. The devices and method may be used for determining an analysis model for predicting an expanded disability status scale (EDSS) indicative of multiple sclerosis, a forced vital capacity indicative of spinal muscular atrophy, or a total motor score (TMS) indicative of Huntington's disease.

### BACKGROUND ART

Disease and, in particular, neurological diseases require an intensive diagnostic measures for disease management. After the onset of the disease, these diseases, typically, are progressive diseases and need to be evaluated by a staging system in order to determine the precise status. Prominent examples among those progressive neurological diseases are multiple sclerosis (MS), Huntington's Disease (HD) and spinal muscular atrophy (SMA).

Currently, the staging of such disease requires great efforts and is cumbersome for the patients which need to go to medical specialists in hospitals or doctor's offices. Moreover, staging requires experience at the end of the medical specialist and is often subjective and based on personal experience and judgement. Nevertheless, there are some parameters from disease staging which are particularly useful for the disease management. Moreover, there are other cases such as in SMA were a clinically relevant parameter such as the forced vital capacity needs to be determined by special equipment, i.e. spirometric devices. For all of these cases, it might be helpful to determine surrogates. Suitable surrogates include biomarkers and, in particular, digitally acquired biomarkers such as performance parameters from tests which aim at determining performance parameters of biological functions that can be correlated to the staging systems or that can be surrogate markers for the clinical parameters.

Correlations between the actual clinical parameter of interest, such as a score or other clinical parameter, can be derived from data by various analysis methods. Based on these methods, models can be established which allow for predicting the actual clinical parameter value based on the surrogate markers which are fed into the model. However, it is decisive to identify and apply a model which shows the best correlation and, thus, yields the best prediction for the clinical parameters.

WO 2018/132483 A1 describes example systems, methods, and apparatus for using data collected from the responses of an individual with the computerized tasks of a cognitive platform to derive performance metrics as an indicator of cognitive abilities, and applying predictive models to the performance metrics and data indicative of one or both of the individual's age and gender to generate an indication of neurodegenerative condition.

CN 109 717 833 A describes a neurological disease auxiliary diagnosis system based on human body motion postures and belongs to the field of intelligent medical treatment. The neurological disease auxiliary diagnosis system quantifies motion postures of subjects to be examined, extracts 23-dimensional gait related features from human body motion posture data, inputs the related features into a classification prediction model to diagnose the subjects to be examined, generates a visual motion function examination report for results of diagnosis of the subjects to be examined, and provides an auxiliary diagnosis suggestion.

US 2017/308981 A1 describes a computer-implemented method which identifies a risk of developing a condition for a particular patient. First, an initial variable set is developed by utilizing one or more patient databases. Second, a model predictive of a selected condition is created using machine learning. With the model developed, patient features vectors are created from a patient health information database for the initial variable set. The model is applied to these patient features vectors to predict development of the condition. Patients predicted to have the condition can be enrolled in an appropriate intervention program.

US 2016/192889 A1 describes a method and a system for an adaptive pattern recognition for psychosis risk modeling with at least the following steps and features: automatically generating a first risk quantification or classification system on the basis of brain images and data mining; automatically generating a second risk quantification or classification system on the basis of genomic and/or metabolomic information and data mining and further processing the first and second risk quantification or classification systems by data mining computing so as to create a meta-level risk quantification data to automatically quantify psychosis risk at the single-subject level.

There is a need for automatically building of models that can analyze large amount of data and complex data and which deliver fast, reliable and accurate results.

### Problem to be Solved

It is therefore desirable to provide methods and devices which address the above-mentioned technical challenges. Specifically, devices and methods for determining at least one analysis model for predicting at least one target variable indicative of a disease status shall be provided which ensure fast and automatically building of a reliable and disease specific analysis model.

### SUMMARY

This problem is addressed by a machine learning system for determining at least one analysis model for predicting at least one target variable indicative of a disease status, a computer-implemented method for determining at least one analysis model for predicting at least one target variable indicative of a disease status, a computer program and uses with the features of the independent claims. Advantageous embodiments which might be realized in an isolated fashion or in any arbitrary combinations are listed in the dependent claims.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a machine learning system for determining at least one analysis model for predicting at least one target variable indicative of a disease status is proposed.

The machine learning system comprises:
at least one communication interface configured for receiving input data, wherein the input data comprises a set of historical digital biomarker feature data, wherein the set of historical digital biomarker feature data comprises a plurality of measured values indicative of the disease status to be predicted;
at least one model unit comprising at least one machine learning model comprising at least one algorithm;
at least one processing unit, wherein the processing unit is configured for determining at least one training data set and at least one test data set from the input data set, wherein the processing unit is configured for determining the analysis model by training the machine learning model with the training data set, wherein the processing unit is configured for predicting the target variable on the test data set using the determined analysis model, wherein the processing unit is configured for determining performance of the determined analysis model based on the predicted target variable and a true value of the target variable of the test data set.

The term “machine learning” as used herein is a broad term and is to be given its ordinary and customary meaning to a person of ordinary skill in the art and is not to be limited to a special or customized meaning. The term specifically may refer, without limitation, to a method of using artificial intelligence (AI) for automatically model building of analytical models. The term “machine learning system” as used herein is a broad term and is to be given its ordinary and customary meaning to a person of ordinary skill in the art and is not to be limited to a special or customized meaning. The term specifically may refer, without limitation, to a system comprising at least one processing unit such as a processor, microprocessor, or computer system configured for machine learning, in particular for executing a logic in a given algorithm. The machine learning system may be configured for performing and/or executing at least one machine learning algorithm, wherein the machine learning algorithm is configured for building the at least one analysis model based on the training data.

The term “analysis model” as used herein is a broad term and is to be given its ordinary and customary meaning to a person of ordinary skill in the art and is not to be limited to a special or customized meaning. The term specifically may refer, without limitation, to a mathematical model configured for predicting at least one target variable for at least one state variable. The analysis model may be a regression model or a classification model. The term “regression model” as used herein is a broad term and is to be given its ordinary and customary meaning to a person of ordinary skill in the art and is not to be limited to a special or customized meaning. The term specifically may refer, without limitation, to an analysis model comprising at least one supervised learning algorithm having as output a numerical value within a range. The term “classification model” as used herein is a broad term and is to be given its ordinary and customary meaning to a person of ordinary skill in the art and is not to be limited to a special or customized meaning. The term specifically may refer, without limitation, to an analysis model comprising at least one supervised learning algorithm having as output a classifier such as “ill” or “healthy”.

The term “target variable” as used herein is a broad term and is to be given its ordinary and customary meaning to a person of ordinary skill in the art and is not to be limited to a special or customized meaning. The term specifically may refer, without limitation, to a clinical value which is to be predicted. The target variable value which is to be predicted may dependent on the disease whose presence or status is to be predicted. The target variable may be either numerical or categorical. For example, the target variable may be categorical and may be “positive” in case of presence of disease or “negative” in case of absence of the disease.

The target variable may be numerical such as at least one value and/or scale value.

For example, the disease whose status is to be predicted is multiple sclerosis. The term “multiple sclerosis (MS)” as used herein relates to disease of the central nervous system (CNS) that typically causes prolonged and severe disability in a subject suffering therefrom. There are four standardized subtype definitions of MS which are also encompassed by the term as used in accordance with the present invention: relapsing-remitting, secondary progressive, primary progressive and progressive relapsing. The term relapsing forms of MS is also used and encompasses relapsing-remitting and secondary progressive MS with superimposed relapses. The relapsing-remitting subtype is characterized by unpredictable relapses followed by periods of months to years of remission with no new signs of clinical disease activity. Deficits suffered during attacks (active status) may either resolve or leave sequelae. This describes the initial course of 85 to 90% of subjects suffering from MS. Secondary progressive MS describes those with initial relapsing-remitting MS, who then begin to have progressive neurological decline between acute attacks without any definite periods of remission. Occasional relapses and minor remissions may appear. The median time between disease onset and conversion from relapsing remitting to secondary progressive MS is about 19 years. The primary progressive subtype describes about 10 to 15% of subjects who never have remission after their initial MS symptoms. It is characterized by progressive of disability from onset, with no, or only occasional and minor, remissions and improvements. The age of onset for the primary progressive subtype is later than other subtypes. Progressive relapsing MS describes those subjects who, from onset, have a steady neurological decline but also suffer clear superimposed attacks. It is now accepted that this latter progressive relapsing phenotype is a variant of primary progressive MS (PPMS) and diagnosis of PPMS according to McDonald 2010 criteria includes the progressive relapsing variant.

Symptoms associated with MS include changes in sensation (hypoesthesia and par-aesthesia), muscle weakness, muscle spasms, difficulty in moving, difficulties with co-ordination and balance (ataxia), problems in speech (dysarthria) or swallowing (dysphagia), visual problems (nystagmus, optic neuritis and reduced visual acuity, or diplopia), fatigue, acute or chronic pain, bladder, sexual and bowel difficulties. Cognitive impairment of varying degrees as well as emotional symptoms of depression or unstable mood are also frequent symptoms. The main clinical measure of disability progression and symptom severity is the Expanded Disability Status Scale (EDSS). Further symptoms of MS are well known in the art and are described in the standard text books of medicine and neurology.

The term “progressing MS” as used herein refers to a condition, where the disease and/or one or more of its symptoms get worse over time. Typically, the progression is accompanied by the appearance of active statuses. The said progression may occur in all subtypes of the disease. However, typically “progressing MS” shall be determined in accordance with the present invention in subjects suffering from relapsing-remitting MS.

Determining status of multiple sclerosis, generally comprises assessing at least one symptom associated with multiple sclerosis selected from a group consisting of: impaired fine motor abilities, pins and needles, numbness in the fingers, fatigue and changes to diurnal rhythms, gait problems and walking difficulty, cognitive impairment including problems with processing speed. Disability in multiple sclerosis may be quantified according to the expanded disability status scale (EDSS) as described in Kurtzke J F, “Rating neurologic impairment in multiple sclerosis: an expanded disability status scale (EDSS)”, November 1983, Neurology. 33 (11): 1444-52. doi:10.1212/WNL.33.11.1444. PMID 6685237. The target variable may be an EDSS value.

The term “expanded disability status scale (EDSS)” as used herein, thus, refers to a score based on quantitative assessment of the disabilities in subjects suffering from MS (Krutzke 1983). The EDSS is based on a neurological examination by a clinician. The EDSS quantifies disability in eight functional systems by assigning a Functional System Score (FSS) in each of these functional systems. The functional systems are the pyramidal system, the cerebellar system, the brainstem system, the sensory system, the bowel and bladder system, the visual system, the cerebral system and other (remaining) systems. EDSS steps 1.0 to 4.5 refer to subjects suffering from MS who are fully ambulatory, EDSS steps 5.0 to 9.5 characterize those with impairment to ambulation.

The clinical meaning of each possible result is the following:
0.0: Normal Neurological Exam
1.0: No disability, minimal signs in 1 FS
1.5: No disability, minimal signs in more than 1 FS
2.0: Minimal disability in 1 FS
2.5: Mild disability in 1 or Minimal disability in 2 FS
3.0: Moderate disability in 1 FS or mild disability in 3-4 FS, though fully ambulatory
3.5: Fully ambulatory but with moderate disability in 1 FS and mild disability in 1 or
2 FS; or moderate disability in 2 FS; or mild disability in 5 FS
4.0: Fully ambulatory without aid, up and about 12 hrs a day despite relatively severe disability. Able to walk without aid 500 meters
4.5: Fully ambulatory without aid, up and about much of day, able to work a full day, may otherwise have some limitations of full activity or require minimal assistance. Relatively severe disability. Able to walk without aid 300 meters
5.0: Ambulatory without aid for about 200 meters. Disability impairs full daily activities
5.5: Ambulatory for 100 meters, disability precludes full daily activities
6.0: Intermittent or unilateral constant assistance (cane, crutch or brace) required to walk 100 meters with or without resting
6.5: Constant bilateral support (cane, crutch or braces) required to walk 20 meters without resting
7.0: Unable to walk beyond 5 meters even with aid, essentially restricted to wheelchair, wheels self, transfers alone; active in wheelchair about 12 hours a day
7.5: Unable to take more than a few steps, restricted to wheelchair, may need aid to transfer; wheels self, but may require motorized chair for full day's activities
8.0: Essentially restricted to bed, chair, or wheelchair, but may be out of bed much of day; retains self-care functions, generally effective use of arms
8.5: Essentially restricted to bed much of day, some effective use of arms, retains some self-care functions
9.0: Helpless bed patient, can communicate and eat
9.5: Unable to communicate effectively or eat/swallow
10.0: Death due to MS

For example, the disease whose status is to be predicted is spinal muscular atrophy.

The term “spinal muscular atrophy (SMA)” as used herein relates to a neuromuscular disease which is characterized by the loss of motor neuron function, typically, in the spinal cord. As a consequence of the loss of motor neuron function, typically, muscle atrophy occurs resulting in an early death of the affected subjects. The disease is caused by an inherited genetic defect in the SMN1 gene. The SMN protein encoded by said gene is required for motor neuron survival. The disease is inherited in an autosomal recessive manner.

Symptoms associated with SMA include areflexia, in particular, of the extremities, muscle weakness and poor muscle tone, difficulties in completing developmental phases in childhood, as a consequence of weakness of respiratory muscles, breathing problems occurs as well as secretion accumulation in the lung, as well as difficulties in sucking, swallowing and feeding/eating. Four different types of SMA are known.

The infantile SMA or SMA1 (Werdnig-Hoffmann disease) is a severe form that manifests in the first months of life, usually with a quick and unexpected onset (“floppy baby syndrome”). A rapid motor neuron death causes inefficiency of the major body organs, in particular, of the respiratory system, and pneumonia-induced respiratory failure is the most frequent cause of death. Unless placed on mechanical ventilation, babies diagnosed with SMA1 do not generally live past two years of age, with death occurring as early as within weeks in the most severe cases, sometimes termed SMA0. With proper respiratory support, those with milder SMA1 phenotypes accounting for around 10% of SMA1 cases are known to live into adolescence and adulthood.

The intermediate SMA or SMA2 (Dubowitz disease) affects children who are never able to stand and walk but who are able to maintain a sitting position at least some time in their life. The onset of weakness is usually noticed some time between 6 and 18 months. The progress is known to vary. Some people gradually grow weaker over time while others through careful maintenance avoid any progression. Scoliosis may be present in these children, and correction with a brace may help improve respiration. Muscles are weakened, and the respiratory system is a major concern. Life expectancy is somewhat reduced but most people with SMA2 live well into adulthood.

The juvenile SMA or SMA3 (Kugelberg-Welander disease) manifests, typically, after 12 months of age and describes people with SMA3 who are able to walk without support at some time, although many later lose this ability. Respiratory involvement is less noticeable, and life expectancy is normal or near normal.

The adult SMA or SMA4 manifests, usually, after the third decade of life with gradual weakening of muscles that affects proximal muscles of the extremities frequently requiring the person to use a wheelchair for mobility. Other complications are rare, and life expectancy is unaffected.

Typically, SMA in accordance with the present invention is SMA1 (Werdnig-Hoffmann disease), SMA2 (Dubowitz disease), SMA3 (Kugelberg-Welander diseases) or SMA4 SMA is typically diagnosed by the presence of the hypotonia and the absence of reflexes. Both can be measured by standard techniques by the clinician in a hospital including electromyography. Sometimes, serum creatine kinase may be increased as a biochemical parameter. Moreover, genetic testing is also possible, in particular, as prenatal diagnostics or carrier screening. Moreover, a critical parameter in SMA management is the function of the respiratory system. The function of the respiratory system can be, typically, determined by measuring the forced vital capacity of the subject which will be indicative for the degree of impairment of the respiratory system as a consequence of SMA.

The term “forced vital capacity (FVC)” as used herein refers to is the volume in liters of air that can forcibly be blown out after full inspiration by a subject. It is, typically, determined by spirometry in a hospital or at a doctor's residency using spirometric devices.

Determining status of spinal muscular atrophy, generally comprises assessing at least one symptom associated with spinal muscular atrophy selected from a group consisting of: hypotonia and muscle weakness, fatigue and changes to diurnal rhythms. A measure for status of spinal muscular atrophy may be the Forced vital capacity (FVC). The FVC may be a quantitative measure for volume of air that can forcibly be blown out after full inspiration, measured in liters, see https://en.wikipedia.org/wiki/Spirometry. The target variable may be a FVC value.

For example, the disease whose status is to be predicted is Huntington's disease. The term “Huntington's Disease (HD)” as used herein relates to an inherited neurological disorder accompanied by neuronal cell death in the central nervous system. Most prominently, the basal ganglia are affected by cell death. There are also further areas of the brain involved such as substantia nigra, cerebral cortex, hippocampus and the purkinje cells. All regions, typically, play a role in movement and behavioral control. The disease is caused by genetic mutations in the gene encoding Huntingtin. Huntingtin is a protein involved in various cellular functions and interacts with over 100 other proteins. The mutated Huntingtin appears to be cytotoxic for certain neuronal cell types. Mutated Huntingtin is characterized by a poly glutamine region caused by a trinucleotide repeat in the Huntingtin gene. A repeat of more than 36 glutamine residues in the poly glutamine region of the protein results in the disease causing Huntingtin protein.

The symptoms of the disease most commonly become noticeable in the mid-age, but can begin at any age from infancy to the elderly. In early stages, symptoms involve subtle changes in personality, cognition, and physical skills. The physical symptoms are usually the first to be noticed, as cognitive and behavioral symptoms are generally not severe enough to be recognized on their own at said early stages. Almost everyone with HD eventually exhibits similar physical symptoms, but the onset, progression and extent of cognitive and behavioral symptoms vary significantly between individuals. The most characteristic initial physical symptoms are jerky, random, and uncontrollable movements called chorea. Chorea may be initially exhibited as general restlessness, small unintentionally initiated or uncompleted motions, lack of coordination, or slowed saccadic eye movements. These minor motor abnormalities usually precede more obvious signs of motor dysfunction by at least three years. The clear appearance of symptoms such as rigidity, writhing motions or abnormal posturing appear as the disorder progresses. These are signs that the system in the brain that is responsible for movement has been affected. Psychomotor functions become increasingly impaired, such that any action that requires muscle control is affected. Common consequences are physical instability, abnormal facial expression, and difficulties chewing, swallowing, and speaking. Consequently, eating difficulties and sleep disturbances are also accompanying the disease. Cognitive abilities are also impaired in a progressive manner. Impaired are executive functions, cognitive flexibility, abstract thinking, rule acquisition, and proper action/reaction capabilities. In more pronounced stages, memory deficits tend to appear including short-term memory deficits to long-term memory difficulties. Cognitive problems worsen over time and will ultimately turn into dementia. Psychiatric complications accompanying HD are anxiety, depression, a reduced display of emotions (blunted affect), egocentrism, aggression, and compulsive behavior, the latter of which can cause or worsen addictions, including alcoholism, gambling, and hypersexuality.

There is no cure for HD. There are supportive measurements in disease management depending on the symptoms to be addressed. Moreover, a number of drugs are used to ameliorate the disease, its progression or the symptoms accompanying it. Tetrabenazine is approved for treatment of HD, include neuroleptics and benzodiazepines are used as drugs that help to reduce chorea, amantadine or remacemide are still under investigation but have shown preliminary positive results. Hypokinesia and rigidity, especially in juvenile cases, can be treated with antiparkinsonian drugs, and myoclonic hyperkinesia can be treated with valproic acid. Ethyl-eicosapentoic acid was found to enhance the motor symptoms of patients, however, its long-term effects need to be revealed.

The disease can be diagnosed by genetic testing. Moreover, the severity of the disease can be staged according to Unified Huntington's Disease Rating Scale (UHDRS). This scale system addresses four components, i.e. the motor function, the cognition, behavior and functional abilities. The motor function assessment includes assessment of ocular pursuit, saccade initiation, saccade velocity, dysarthria, tongue protrusion, maximal dystonia, maximal chorea, retropulsion pull test, finger taps, pronate/supinate hands, luria, rigidity arms, bradykinesia body, gait, and tandem walking and can be summarized as total motor score (TMS). The motoric functions must be investigated and judged by a medical practitioner.

Determining status of Huntington's disease generally comprises assessing at least one symptom associated with Huntington's disease selected from a group consisting of: Psychomotor slowing, chorea (jerking, writhing), progressive dysarthria, rigidity and dystonia, social withdrawal, progressive cognitive impairment of processing speed, attention, planning, visual-spatial processing, learning (though intact recall), fatigue and changes to diurnal rhythms. A measure for status of is a total motor score (TMS). The target variable may be a total motor score (TMS) value. The term “total motor score (TMS)” as used herein, thus, refers to a score based on assessment of ocular pursuit, saccade initiation, saccade velocity, dysarthria, tongue protrusion, maximal dystonia, maximal chorea, retropulsion pull test, finger taps, pronate/supinate hands, luria, rigidity arms, bradykinesia body, gait, and tandem walking.

The term “state variable” as used herein is a broad term and is to be given its ordinary and customary meaning to a person of ordinary skill in the art and is not to be limited to a special or customized meaning. The term specifically may refer, without limitation, to an input variable which can be filled in the prediction model such as data derived by medical examination and/or self-examination by a subject. The state variable may be determined in at least one active test and/or in at least one passive monitoring. For example, the state variable may be determined in an active test such as at least one cognition test and/or at least one hand motor function test and/or or at least one mobility test.

The term “subject” as used herein, typically, relates to mammals. The subject in accordance with the present invention may, typically, suffer from or shall be suspected to suffer from a disease, i.e. it may already show some or all of the negative symptoms associated with the said disease. In an embodiment of the invention said subject is a human.

The state variable may be determined by using at least one mobile device of the subject. The term “mobile device” as used herein is a broad term and is to be given its ordinary and customary meaning to a person of ordinary skill in the art and is not to be limited to a special or customized meaning. The term may specifically refer, without limitation, to a mobile electronics device, more specifically to a mobile communication device comprising at least one processor. The mobile device may specifically be a cell phone or smartphone. The mobile device may also refer to a tablet computer or any other type of portable computer. The mobile device may comprise a data acquisition unit which may be configured for data acquisition. The mobile device may be configured for detecting and/or measuring either quantitatively or qualitatively physical parameters and transform them into electronic signals such as for further processing and/or analysis. For this purpose, the mobile device may comprise at least one sensor. It will be understood that more than one sensor can be used in the mobile device, i.e. at least two, at least three, at least four, at least five, at least six, at least seven, at least eight, at least nine or at least ten or even more different sensors. The sensor may be at least one sensor selected from the group consisting of: at least one gyroscope, at least one magnetometer, at least one accelerometer, at least one proximity sensor, at least one thermometer, at least one pedometer, at least one fingerprint detector, at least one touch sensor, at least one voice recorder, at least one light sensor, at least one pressure sensor, at least one location data detector, at least one camera, at least one GPS, and the like. The mobile device may comprise the processor and at least one database as well as software which is tangibly embedded to said device and, when running on said device, carries out a method for data acquisition. The mobile device may comprise a user interface, such as a display and/or at least one key, e.g. for performing at least one task requested in the method for data acquisition.

The term “predicting” as used herein is a broad term and is to be given its ordinary and customary meaning to a person of ordinary skill in the art and is not to be limited to a special or customized meaning. The term specifically may refer, without limitation, to determining at least one numerical or categorical value indicative of the disease status for the at least one state variable. In particular, the state variable may be filled in the analysis as input and the analysis model may be configured for performing at least one analysis on the state variable for determining the at least one numerical or categorical value indicative of the disease status. The analysis may comprise using the at least one trained algorithm.

The term “determining at least one analysis model” as used herein is a broad term and is to be given its ordinary and customary meaning to a person of ordinary skill in the art and is not to be limited to a special or customized meaning. The term specifically may refer, without limitation, to building and/or creating the analysis model.

The term “disease status” as used herein is a broad term and is to be given its ordinary and customary meaning to a person of ordinary skill in the art and is not to be limited to a special or customized meaning. The term specifically may refer, without limitation, to health condition and/or medical condition and/or disease stage. For example, the disease status may be healthy or ill and/or presence or absence of disease. For example, the disease status may be a value relating to a scale indicative of disease stage. The term “indicative of a disease status” as used herein is a broad term and is to be given its ordinary and customary meaning to a person of ordinary skill in the art and is not to be limited to a special or customized meaning. The term specifically may refer, without limitation, to information directly relating to the disease status and/or to information indirectly relating to the disease status, e.g. information which need further analysis and/or processing for deriving the disease status. For example, the target variable may be a value which need to be compared to a table and/or lookup table for determine the disease status.

The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. In particular, the communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may specifically provide means for transferring or exchanging information. In particular, the communication interface may provide a data transfer connection, e.g. Bluetooth, NFC, inductive coupling or the like. As an example, the communication interface may be or may comprise at least one port comprising one or more of a network or internet port, a USB-port and a disk drive. The communication interface may be at least one web interface.

The term “input data” as used herein is a broad term and is to be given its ordinary and customary meaning to a person of ordinary skill in the art and is not to be limited to a special or customized meaning. The term specifically may refer, without limitation, to experimental data used for model building. The input data comprises the set of historical digital biomarker feature data. The term “biomarker” as used herein is a broad term and is to be given its ordinary and customary meaning to a person of ordinary skill in the art and is not to be limited to a special or customized meaning. The term specifically may refer, without limitation, to a measurable characteristic of a biological state and/or biological condition. The term “feature” as used herein is a broad term and is to be given its ordinary and customary meaning to a person of ordinary skill in the art and is not to be limited to a special or customized meaning. The term specifically may refer, without limitation, to a measurable property and/or characteristic of a symptom of the disease on which the prediction is based. In particular, all features from all tests may be considered and the optimal set of features for each prediction is determined. Thus, all features may be considered for each disease. The term “digital biomarker feature data” as used herein is a broad term and is to be given its ordinary and customary meaning to a person of ordinary skill in the art and is not to be limited to a special or customized meaning. The term specifically may refer, without limitation, to experimental data determined by at least one digital device such as by a mobile device which comprises a plurality of different measurement values per subject relating to symptoms of the disease. The digital biomarker feature data may be determined by using at least one mobile device. With respect to the mobile device and determining of digital biomarker feature data with the mobile device reference is made to the description of the determination of the state variable with the mobile device above. The set of historical digital biomarker feature data comprises a plurality of measured values per subject indicative of the disease status to be predicted. The term “historical” as used herein is a broad term and is to be given its ordinary and customary meaning to a person of ordinary skill in the art and is not to be limited to a special or customized meaning. The term specifically may refer, without limitation, to the fact that the digital biomarker feature data was determined and/or collected before model building such as during at least one test study. For example, for model building for predicting at least one target indicative of multiple sclerosis the digital biomarker feature data may be data from the Floodlight POC study. For example, for model building for predicting at least one target indicative of spinal muscular atrophy the digital biomarker feature data may be data from the OLEOS study. For example, for model building for predicting at least one target indicative of Huntington's disease the digital biomarker feature data may be data from the HD OLE study, ISIS 44319-CS2. The input data may be determined in at least one active test and/or in at least one passive monitoring. For example, the input data may be determined in an active test using at least one mobile device such as at least one cognition test and/or at least one hand motor function test and/or or at least one mobility test.

The input data further may comprise target data. The term “target data” as used herein is a broad term and is to be given its ordinary and customary meaning to a person of ordinary skill in the art and is not to be limited to a special or customized meaning. The term specifically may refer, without limitation, to data comprising clinical values to predict, in particular one clinical value per subject. The target data may be either numerical or categorical. The clinical value may directly or indirectly refer to the status of the disease.

The processing unit may be configured for extracting features from the input data. The term “extracting features” as used herein is a broad term and is to be given its ordinary and customary meaning to a person of ordinary skill in the art and is not to be limited to a special or customized meaning. The term specifically may refer, without limitation, to at least one process of determining and/or deriving features from the input data. Specifically, the features may be pre-defined, and a subset of features may be selected from an entire set of possible features. The extracting of features may comprise one or more of data aggregation, data reduction, data transformation and the like. The processing unit may be configured for ranking the features. The term “ranking features” as used herein is a broad term and is to be given its ordinary and customary meaning to a person of ordinary skill in the art and is not to be limited to a special or customized meaning. The term specifically may refer, without limitation, to assigning a rank, in particular a weight, to each of the features depending on predefined criteria. For example, the features may be ranked with respect to their relevance, i.e. with respect to correlation with the target variable, and/or the features may be ranked with respect to redundancy, i.e. with respect to correlation between features. The processing unit may be configured for ranking the features by using a maximum-relevance-minimum-redundancy technique. This method ranks all features using a trade-off between relevance and redundancy. Specifically, the feature selection and ranking may be performed as described in Ding C., Peng H. “Minimum redundancy feature selection from microarray gene expression data”, J Bioinform Comput Biol. 2005 April; 3 (2):185-205, PubMed PMID:15852500. The feature selection and ranking may be performed by using a modified method compared to the method described in Ding et al. The maximum correlation coefficient may be used rather than the mean correlation coefficient and an addition transformation may be applied to it. In case of a regression model as analysis model the transformation the value of the mean correlation coefficient may be raised to the 5th power. In case of a classification model as analysis model the value of the mean correlation coefficient may be multiplied by 10.

The term “model unit” as used herein is a broad term and is to be given its ordinary and customary meaning to a person of ordinary skill in the art and is not to be limited to a special or customized meaning. The term specifically may refer, without limitation, to at least one data storage and/or storage unit configured for storing at least one machine learning model. The term “machine learning model” as used herein is a broad term and is to be given its ordinary and customary meaning to a person of ordinary skill in the art and is not to be limited to a special or customized meaning. The term specifically may refer, without limitation, to at least one trainable algorithm. The model unit may comprise a plurality of machine learning models, e.g. different machine learning models for building the regression model and machine learning models for building the classification model. For example, the analysis model may be a regression model and the algorithm of the machine learning model may be at least one algorithm selected from the group consisting of: k nearest neighbors (kNN); linear regression; partial last-squares (PLS); random forest (RF); and extremely randomized Trees (XT). For example, the analysis model may be a classification model and the algorithm of the machine learning model may be at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

The term “processing unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing operations of a computer or system, and/or, generally, to a device which is configured for performing calculations or logic operations. The processing unit may comprise at least one processor. In particular, the processing unit may be configured for processing basic instructions that drive the computer or system. As an example, the processing unit may comprise at least one arithmetic logic unit (ALU), at least one floating-point unit (FPU), such as a math coprocessor or a numeric coprocessor, a plurality of registers and a memory, such as a cache memory. In particular, the processing unit may be a multi-core processor. The processing unit may be configured for machine learning. The processing unit may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The processing unit may be configured for pre-processing the input data. The pre-processing may comprise at least one filtering process for input data fulfilling at least one quality criterion. For example, the input data may be filtered to remove missing variables. For example, the pre-processing may comprise excluding data from subjects with less than a pre-defined minimum number of observations.

The term “training data set” as used herein is a broad term and is to be given its ordinary and customary meaning to a person of ordinary skill in the art and is not to be limited to a special or customized meaning. The term specifically may refer, without limitation, to a subset of the input data used for training the machine learning model. The term “test data set” as used herein is a broad term and is to be given its ordinary and customary meaning to a person of ordinary skill in the art and is not to be limited to a special or customized meaning. The term specifically may refer, without limitation, to another subset of the input data used for testing the trained machine learning model. The training data set may comprise a plurality of training data sets. In particular, the training data set comprises a training data set per subject of the input data. The test data set may comprise a plurality of test data sets. In particular, the test data set comprises a test data set per subject of the input data. The processing unit may be configured for generating and/or creating per subject of the input data a training data set and a test data set, wherein the test data set per subject may comprise data only of that subject, whereas the training data set for that subject comprises all other input data.

The processing unit may be configured for performing at least one data aggregation and/or data transformation on both of the training data set and the test data set for each subject. The transformation and feature ranking steps may be performed without splitting into training data set and test data set. This may allow to enable interference of e.g. important feature from the data.

The processing unit may be configured for one or more of at least one stabilizing transformation; at least one aggregation; and at least one normalization for the training data set and for the test data set.

For example, the processing unit may be configured for subject-wise data aggregation of both of the training data set and the test data set, wherein a mean value of the features is determined for each subject.

For example, the processing unit may be configured for variance stabilization, wherein for each feature at least one variance stabilizing function is applied. The variance stabilizing function may be at least one function selected from the group consisting of: a logistic, which may be used if all values are greater 300 and no values are between 0 and 1; a logit, which may be used if all values are between 0 and 1, inclusive; a sigmoid; a log 10, which may be used if considered when all values≥=0. The processing unit may be configured for transforming values of each feature using each of the variance transformation functions. The processing unit may be configured for evaluating each of the resulting distributions, including the original one, using a certain criterion. In case of a classification model as analysis model, i.e. when the target variable is discrete, said criterion may be to what extent the obtained values are able to separate the different classes. Specifically, the maximum of all class-wise mean silhouette values may be used for this end. In case of a regression model as analysis model, the criterion may be a mean absolute error obtained after regression of values, which were obtained by applying the variance stabilizing function, against the target variable. Using this selection criterion, processing unit may be configured for determining the best possible transformation, if any are better than the original values, on the training data set. The best possible transformation can be subsequently applied to the test data set.

For example, the processing unit may be configured for z-score transformation, wherein for each transformed feature the mean and standard deviations are determined on the training data set, wherein these values are used for z-score transformation on both the training data set and the test data set.

For example, the processing unit may be configured for performing three data transformation steps on both the training data set and the test data set, wherein the transformation steps comprise: 1. subject-wise data aggregation; 2. variance stabilization; 3. z-score transformation.

The processing unit may be configured for determining and/or providing at least one output of the ranking and transformation steps. For example, the output of the ranking and transformation steps may comprise at least one diagnostics plots. The diagnostics plot may comprise at least one principal component analysis (PCA) plot and/or at least one pair plot comparing key statistics related to the ranking procedure.

The processing unit is configured for determining the analysis model by training the machine learning model with the training data set. The term “training the machine learning model” as used herein is a broad term and is to be given its ordinary and customary meaning to a person of ordinary skill in the art and is not to be limited to a special or customized meaning. The term specifically may refer, without limitation, to a process of determining parameters of the algorithm of machine learning model on the training data set. The training may comprise at least one optimization or tuning process, wherein a best parameter combination is determined. The training may be performed iteratively on the training data sets of different subjects. The processing unit may be configured for considering different numbers of features for determining the analysis model by training the machine learning model with the training data set. The algorithm of the machine learning model may be applied to the training data set using a different number of features, e.g. depending on their ranking. The training may comprise n-fold cross validation to get a robust estimate of the model parameters. The training of the machine learning model may comprise at least one controlled learning process, wherein at least one hyper-parameter is chosen to control the training process. If necessary the training is step is repeated to test different combinations of hyper-parameters.

In particular subsequent to the training of the machine learning model, the processing unit is configured for predicting the target variable on the test data set using the determined analysis model. The term “determined analysis model” as used herein is a broad term and is to be given its ordinary and customary meaning to a person of ordinary skill in the art and is not to be limited to a special or customized meaning. The term specifically may refer, without limitation, to the trained machine learning model. The processing unit may be configured for predicting the target variable for each subject based on the test data set of that subject using the determined analysis model. The processing unit may be configured for predicting the target variable for each subject on the respective training and test data sets using the analysis model. The processing unit may be configured for recording and/or storing both the predicted target variable per subject and the true value of the target variable per subject, for example, in at least one output file. The term “true value of the target variable” as used herein is a broad term and is to be given its ordinary and customary meaning to a person of ordinary skill in the art and is not to be limited to a special or customized meaning. The term specifically may refer, without limitation, to the real or actual value of the target variable of that subject, which may be determined from the target data of that subject.

The processing unit is configured for determining performance of the determined analysis model based on the predicted target variable and the true value of the target variable of the test data set. The term “performance” as used herein is a broad term and is to be given its ordinary and customary meaning to a person of ordinary skill in the art and is not to be limited to a special or customized meaning. The term specifically may refer, without limitation, to suitability of the determined analysis model for predicting the target variable. The performance may be characterized by deviations between predicted target variable and true value of the target variable. The machine learning system may comprises at least one output interface. The output interface may be designed identical to the communication interface and/or may be formed integral with the communication interface. The output interface may be configured for providing at least one output. The output may comprise at least one information about the performance of the determined analysis model. The information about the performance of the determined analysis model may comprises one or more of at least one scoring chart, at least one predictions plot, at least one correlations plot, and at least one residuals plot.

The model unit may comprise a plurality of machine learning models, wherein the machine learning models are distinguished by their algorithm. For example, for building a regression model the model unit may comprise the following algorithms k nearest neighbors (kNN), linear regression, partial last-squares (PLS), random forest (RF), and extremely randomized Trees (XT). For example, for building a classification model the model unit may comprise the following algorithms k nearest neighbors (kNN), support vector machines (SVM), linear discriminant analysis (LDA), quadratic discriminant analysis (QDA), naïve Bayes (NB), random forest (RF), and extremely randomized Trees (XT). The processing unit may be configured for determining a analysis model for each of the machine learning models by training the respective machine learning model with the training data set and for predicting the target variables on the test data set using the determined analysis models.

The processing unit may be configured for determining performance of each of the determined analysis models based on the predicted target variables and the true value of the target variable of the test data set. In case of building a regression model, the output provided by the processing unit may comprise one or more of at least one scoring chart, at least one predictions plot, at least one correlations plot, and at least one residuals plot. The scoring chart may be a box plot depicting for each subject a mean absolute error from both the test and training data set and for each type of regressor, i.e. the algorithm which was used, and number of features selected. The predictions plot may show for each combination of regressor type and number of features, how well the predicted values of the target variable correlate with the true value, for both the test and the training data. The correlations plot may show the Spearman correlation coefficient between the predicted and true target variables, for each regressor type, as a function of the number of features included in the model. The residuals plot may show the correlation between the predicted target variable and the residual for each combination of regressor type and number of features, and for both the test and training data.

The processing unit may be configured for determining the analysis model having the best per-formance, in particular based on the output.

In case of building a classification model, the output provided by the processing unit may comprise the scoring chart, showing in a box plot for each subject the mean F1 performance score, also denoted as F-score or F-measure, from both the test and training data and for each type of regressor and number of features selected. The processing unit may be configured for determining the analysis model having the best performance, in particular based on the output.

In a further aspect of the present invention, a computer implemented method for determining at least one analysis model for predicting at least one target variable indicative of a disease status is proposed. In the method a machine learning system according to the present invention is used. Thus, with respect to embodiments and definitions of the method reference is made to the description of the machine learning system above or as described in further detail below.

The method comprises the following method steps which, specifically, may be performed in the given order. Still, a different order is also possible. It is further possible to perform two or more of the method steps fully or partially simultaneously. Further, one or more or even all of the method steps may be performed once or may be performed repeatedly, such as repeated once or several times. Further, the method may comprise additional method steps which are not listed.

The method comprises the following steps:
a) receiving input data via at least one communication interface, wherein the input data comprises a set of historical digital biomarker feature data, wherein the set of historical digital biomarker feature data comprises a plurality of measured values indicative of the disease status to be predicted;
   at least one processing unit:
b) determining at least one training data set and at least one test data set from the input data set;
c) determining the analysis model by training a machine learning model comprising at least one algorithm with the training data set;
d) predicting the target variable on the test data set using the determined analysis model;
e) determining performance of the determined analysis model based on the predicted target variable and a true value of the target variable of the test data set.

In step c) a plurality of analysis models may be determined by training a plurality of machine learning models with the training data set. The machine learning models may be distinguished by their algorithm. In step d) a plurality of target variables may be predicted on the test data set using the determined analysis models. In step e) the performance of each of the determined analysis models may be determined based on the predicted target variables and the true value of the target variable of the test data set. The method further may comprise determining the analysis model having the best performance.

Further disclosed and proposed herein is a computer program for determining at least one analysis model for predicting at least one target variable indicative of a disease status including computer-executable instructions for performing the method according to the present invention in one or more of the embodiments enclosed herein when the program is executed on a computer or computer network. Specifically, the computer program may be stored on a computer-readable data carrier and/or on a computer-readable storage medium. The computer program is configured to perform at least steps b) to e) of the method according to the present invention in one or more of the embodiments enclosed herein.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps b) to e) as indicated above may be performed by using a computer or a computer network, preferably by using a computer program.

Further disclosed and proposed herein is a computer program product having program code means, in order to perform the method according to the present invention in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In a further aspect of the present invention a use of a machine learning system according to according to one or more of the embodiments disclosed herein is proposed for predicting one or more of an expanded disability status scale (EDSS) value indicative of multiple sclerosis, a forced vital capacity (FVC) value indicative of spinal muscular atrophy, or a total motor score (TMS) value indicative of Huntington's disease.

The devices and methods according to the present invention have several advantages over known methods for predicting disease status. The use of a machine learning system may allow to analyze large amount of complex input data, such as data determined in several and large test studies, and allow to determine analysis models which allow delivering fast, reliable and accurate results.

Summarizing and without excluding further possible embodiments, the following embodiments may be envisaged:

Embodiment 1: A machine learning system for determining at least one analysis model for predicting at least one target variable indicative of a disease status comprising:
at least one communication interface configured for receiving input data, wherein the input data comprises a set of historical digital biomarker feature data, wherein the set of historical digital biomarker feature data comprises a plurality of measured values indicative of the disease status to be predicted;
at least one model unit comprising at least one machine learning model comprising at least one algorithm;
at least one processing unit, wherein the processing unit is configured for determining at least one training data set and at least one test data set from the input data set, wherein the processing unit is configured for determining the analysis model by training the machine learning model with the training data set, wherein the processing unit is configured for predicting the target variable on the test data set using the determined analysis model, wherein the processing unit is configured for determining performance of the determined analysis model based on the predicted target variable and a true value of the target variable of the test data set.

Embodiment 2: The machine learning system according to the preceding embodiment, wherein the analysis model is a regression model or a classification model.

Embodiment 3: The machine learning system according to the preceding embodiment, wherein the analysis model is a regression model, wherein the algorithm of the machine learning model is at least one algorithm selected from the group consisting of: k nearest neighbors (kNN); linear regression; partial last-squares (PLS); random forest (RF); and extremely randomized Trees (XT), or wherein the analysis model is a classification model, wherein the algorithm of the machine learning model is at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

Embodiment 4: The machine learning system according to any one of the preceding embodiments, wherein the model unit comprises a plurality of machine learning models, wherein the machine learning models are distinguished by their algorithm.

Embodiment 5: The machine learning system according to the preceding embodiment, wherein the processing unit is configured for determining an analysis model for each of the machine learning models by training the respective machine learning model with the training data set and for predicting the target variables on the test data set using the determined analysis models, wherein the processing unit is configured for determining performance of each of the determined analysis models based on the predicted target variables and the true value of the target variable of the test data set, wherein the processing unit is configured for determining the analysis model having the best performance.

Embodiment 6: The machine learning system according to any one of the preceding embodiments, wherein the target variable is a clinical value to be predicted, wherein the target variable is either numerical or categorical.

Embodiment 7: The machine learning system according to any one of the preceding embodiments, wherein the disease whose status is to be predicted is multiple sclerosis and the target variable is an expanded disability status scale (EDSS) value, or wherein the disease whose status is to be predicted is spinal muscular atrophy and the target variable is a forced vital capacity (FVC) value, or wherein the disease whose status is to be predicted is Huntington's disease and the target variable is a total motor score (TMS) value.

Embodiment 8: The machine learning system according to any one of the preceding embodiments, wherein the processing unit is configured for generating and/or creating per subject of the input data a training data set and a test data set, wherein the test data set comprises data of one subject, wherein the training data set comprises the other input data.

Embodiment 9: The machine learning system according to any one of the preceding embodiments, wherein the processing unit is configured for extracting features from the input data, wherein the processing unit is configured for ranking the features by using a maximum-relevance-minimum-redundancy technique.

Embodiment 10: The machine learning system according to the preceding embodiment, wherein the processing unit is configured for considering different numbers of features for determining the analysis model by training the machine learning model with the training data set.

Embodiment 11: The machine learning system according to any one of the preceding embodiments, wherein the processing unit is configured for pre-processing the input data, wherein the pre-processing comprises at least one filtering process for input data fulfilling at least one quality criterion.

Embodiment 12: The machine learning system according to any one of the preceding embodiments, wherein the processing unit is configured for performing one or more of at least one stabilizing transformation; at least one aggregation; and at least one normalization for the training data set and for the test data set.

Embodiment 13: The machine learning system according to any one of the preceding embodiments, wherein the machine learning system comprises at least one output interface, wherein the output interface is configured for providing at least one output, wherein the output comprises at least one information about the performance of the determined analysis model.

Embodiment 14: The machine learning system according to the preceding embodiment, wherein the information about the performance of the determined analysis model comprises one or more of at least one scoring chart, at least one predictions plot, at least one correlations plot, and at least one residuals plot.

Embodiment 15: A computer-implemented method for determining at least one analysis model for predicting at least one target variable indicative of a disease status, wherein in the method a machine learning system according to any one of the preceding embodiments is used, wherein the method comprises the following steps:
a) receiving input data via at least one communication interface, wherein the input data comprises a set of historical digital biomarker feature data, wherein the set of historical digital biomarker feature data comprises a plurality of measured values indicative of the disease status to be predicted;
   at least one processing unit:
b) determining at least one training data set and at least one test data set from the input data set;
c) determining the analysis model by training a machine learning model comprising at least one algorithm with the training data set;
d) predicting the target variable on the test data set using the determined analysis model;
e) determining performance of the determined analysis model based on the predicted target variable and a true value of the target variable of the test data set.

Embodiment 16: The method according to the preceding embodiment, wherein in step c) a plurality of analysis models is determined by training a plurality of machine learning models with the training data set, wherein the machine learning models are distinguished by their algorithm, wherein in step d) a plurality of target variables is predicted on the test data set using the determined analysis models, wherein in step e) the performance of each of the determined analysis models is determined based on the predicted target variables and the true value of the target variable of the test data set, wherein the method further comprises determining the analysis model having the best performance.

Embodiment 17: Computer program for determining at least one analysis model for predicting at least one target variable indicative of a disease status, configured for causing a computer or computer network to fully or partially perform the method for determining at least one analysis model for predicting at least one target variable indicative of a disease status according to any one of the preceding embodiments referring to a method, when executed on the computer or computer network, wherein the computer program is configured to perform at least steps b) to e) of the method for determining at least one analysis model for predicting at least one target variable indicative of a disease status according to any one of the preceding embodiments referring to a method.

Embodiment 18: A computer-readable storage medium comprising instructions which, when executed by a computer or computer network cause to carry out at least steps b) to e) of the method according to any one of the preceding method embodiments.

Embodiment 19: Use of a machine learning system according to any one of the preceding embodiments referring to a machine learning system for determining an analysis model for predicting one or more of an expanded disability status scale (EDSS) value indicative of multiple sclerosis, a forced vital capacity (FVC) value indicative of spinal muscular atrophy, or a total motor score (TMS) value indicative of Huntington's disease.

### BRIEF DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an exemplary embodiment of a machine learning system according to the present invention;

FIG. 2 shows an exemplary embodiment of a computer-implemented method according to the present invention; and

FIG. 3A, FIG. 3B, and FIG. 3C show embodiments of correlations plots for assessment of performance of an analysis model.

### DETAILED DESCRIPTION OF THE EMBODIMENTS

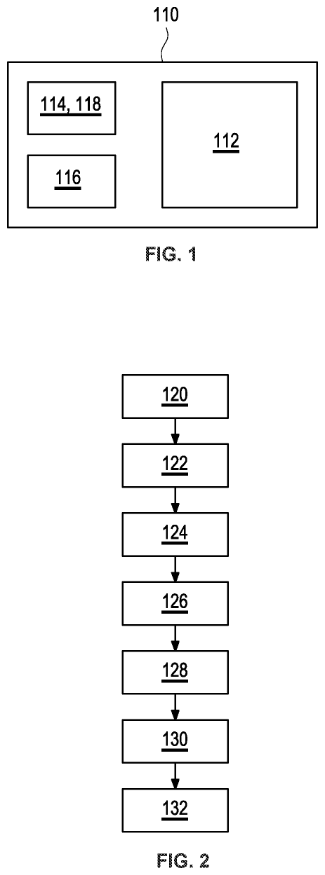

FIG. 1 shows highly schematically an embodiment of a machine learning system 110 for determining at least one analysis model for predicting at least one target variable indicative of a disease status.

The analysis model may be a mathematical model configured for predicting at least one target variable for at least one state variable. The analysis model may be a regression model or a classification model. The regression model may be an analysis model comprising at least one supervised learning algorithm having as output a numerical value within a range. The classification model may be an analysis model comprising at least one supervised learning algorithm having as output a classifier such as “ill” or “healthy”.

The target variable value which is to be predicted may dependent on the disease whose presence or status is to be predicted. The target variable may be either numerical or categorical. For example, the target variable may be categorical and may be “positive” in case of presence of disease or “negative” in case of absence of the disease. The disease status may be a health condition and/or a medical condition and/or a disease stage. For example, the disease status may be healthy or ill and/or presence or absence of disease. For example, the disease status may be a value relating to a scale indicative of disease stage. The target variable may be numerical such as at least one value and/or scale value. The target variable may directly relate to the disease status and/or may indirectly relate to the disease status. For example, the target variable may need further analysis and/or processing for deriving the disease status. For example, the target variable may be a value which need to be compared to a table and/or lookup table for determine the disease status.

The machine learning system 110 comprises at least one processing unit 112 such as a processor, microprocessor, or computer system configured for machine learning, in particular for executing a logic in a given algorithm. The machine learning system 110 may be configured for performing and/or executing at least one machine learning algorithm, wherein the machine learning algorithm is configured for building the at least one analysis model based on the training data. The processing unit 112 may comprise at least one processor. In particular, the processing unit 112 may be configured for processing basic instructions that drive the computer or system. As an example, the processing unit 112 may comprise at least one arithmetic logic unit (ALU), at least one floating-point unit (FPU), such as a math coprocessor or a numeric coprocessor, a plurality of registers and a memory, such as a cache memory. In particular, the processing unit 112 may be a multi-core processor. The processing unit 112 may be configured for machine learning. The processing unit 112 may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The machine learning system comprises at least one communication interface 114 configured for receiving input data. The communication interface 114 may be configured for transferring information from a computational device, e.g. a computer, such as to send or output information, e.g. onto another device. Additionally or alternatively, the communication interface 114 may be configured for transferring information onto a computational device, e.g. onto a computer, such as to receive information. The communication interface 114 may specifically provide means for transferring or exchanging information. In particular, the communication interface 114 may provide a data transfer connection, e.g. Bluetooth, NFC, inductive coupling or the like. As an example, the communication interface 114 may be or may comprise at least one port comprising one or more of a network or internet port, a USB-port and a disk drive. The communication interface 114 may be at least one web interface.

The input data comprises a set of historical digital biomarker feature data, wherein the set of historical digital biomarker feature data comprises a plurality of measured values indicative of the disease status to be predicted. The set of historical digital biomarker feature data comprises a plurality of measured values per subject indicative of the disease status to be predicted. For example, for model building for predicting at least one target indicative of multiple sclerosis the digital biomarker feature data may be data from the Floodlight POC study. For example, for model building for predicting at least one target indicative of spinal muscular atrophy the digital biomarker feature data may be data from the OLEOS study. For example, for model building for predicting at least one target indicative of Huntington's disease the digital biomarker feature data may be data from the HD OLE study, ISIS 44319-CS2. The input data may be determined in at least one active test and/or in at least one passive monitoring. For example, the input data may be determined in an active test using at least one mobile device such as at least one cognition test and/or at least one hand motor function test and/or or at least one mobility test.

The input data further may comprise target data. The target data comprises clinical values to predict, in particular one clinical value per subject. The target data may be either numerical or categorical. The clinical value may directly or indirectly refer to the status of the disease.

The processing unit 112 may be configured for extracting features from the input data. The extracting of features may comprise one or more of data aggregation, data reduction, data transformation and the like. The processing unit 112 may be configured for ranking the features. For example, the features may be ranked with respect to their relevance, i.e. with respect to correlation with the target variable, and/or the features may be ranked with respect to redundancy, i.e. with respect to correlation between features. The processing unit 110 may be configured for ranking the features by using a maximum-relevance-minimum-redundancy technique. This method ranks all features using a trade-off between relevance and redundancy. Specifically, the feature selection and ranking may be performed as described in Ding C., Peng H. “Minimum redundancy feature selection from microarray gene expression data”, J Bioinform Comput Biol. 2005 April; 3 (2):185-205, PubMed PMID:15852500. The feature selection and ranking may be performed by using a modified method compared to the method described in Ding et al. The maximum correlation coefficient may be used rather than the mean correlation coefficient and an addition transformation may be applied to it. In case of a regression model as analysis model the transformation the value of the mean correlation coefficient may be raised to the 5th power. In case of a classification model as analysis model the value of the mean correlation coefficient may be multiplied by 10.

The machine learning system 110 comprises at least one model unit 116 comprising at least one machine learning model comprising at least one algorithm. The model unit 116 may comprise a plurality of machine learning models, e.g. different machine learning models for building the regression model and machine learning models for building the classification model. For example, the analysis model may be a regression model and the algorithm of the machine learning model may be at least one algorithm selected from the group consisting of: k nearest neighbors (kNN); linear regression; partial last-squares (PLS); random forest (RF); and extremely randomized Trees (XT). For example, the analysis model may be a classification model and the algorithm of the machine learning model may be at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

The processing unit 112 may be configured for pre-processing the input data. The pre-processing 112 may comprise at least one filtering process for input data fulfilling at least one quality criterion. For example, the input data may be filtered to remove missing variables.

For example, the pre-processing may comprise excluding data from subjects with less than a pre-defined minimum number of observations.

The processing unit 112 is configured for determining at least one training data set and at least one test data set from the input data set. The training data set may comprise a plurality of training data sets. In particular, the training data set comprises a training data set per subject of the input data. The test data set may comprise a plurality of test data sets. In particular, the test data set comprises a test data set per subject of the input data. The processing unit 112 may be configured for generating and/or creating per subject of the input data a training data set and a test data set, wherein the test data set per subject may comprise data only of that subject, whereas the training data set for that subject comprises all other input data.

The processing unit 112 may be configured for performing at least one data aggregation and/or data transformation on both of the training data set and the test data set for each subject. The transformation and feature ranking steps may be performed without splitting into training data set and test data set. This may allow to enable interference of e.g. important feature from the data. The processing unit 112 may be configured for one or more of at least one stabilizing transformation; at least one aggregation; and at least one normalization for the training data set and for the test data set. For example, the processing unit 112 may be configured for subject-wise data aggregation of both of the training data set and the test data set, wherein a mean value of the features is determined for each subject. For example, the processing unit 112 may be configured for variance stabilization, wherein for each feature at least one variance stabilizing function is applied. The variance stabilizing function may be at least one function selected from the group consisting of: a logistic, which may be used if all values are greater 300 and no values are between 0 and 1; a logit, which may be used if all values are between 0 and 1, inclusive; a sigmoid; a log 10, which may be used if considered when all values≥=0. The processing unit 112 may be configured for transforming values of each feature using each of the variance transformation functions. The processing unit 112 may be configured for evaluating each of the resulting distributions, including the original one, using a certain criterion. In case of a classification model as analysis model, i.e. when the target variable is discrete, said criterion may be to what extent the obtained values are able to separate the different classes. Specifically, the maximum of all class-wise mean silhouette values may be used for this end. In case of a regression model as analysis model, the criterion may be a mean absolute error obtained after regression of values, which were obtained by applying the variance stabilizing function, against the target variable. Using this selection criterion, processing unit 112 may be configured for determining the best possible transformation, if any are better than the original values, on the training data set. The best possible transformation can be subsequently applied to the test data set. For example, the processing unit 112 may be configured for z-score transformation, wherein for each transformed feature the mean and standard deviations are determined on the training data set, wherein these values are used for z-score transformation on both the training data set and the test data set. For example, the processing unit 112 may be configured for performing three data transformation steps on both the training data set and the test data set, wherein the transformation steps comprise: 1. subject-wise data aggregation; 2. variance stabilization; 3. z-score transformation. The processing unit 112 may be configured for determining and/or providing at least one output of the ranking and transformation steps. For example, the output of the ranking and transformation steps may comprise at least one diagnostics plots. The diagnostics plot may comprise at least one principal component analysis (PCA) plot and/or at least one pair plot comparing key statistics related to the ranking procedure.

The processing unit 112 is configured for determining the analysis model by training the machine learning model with the training data set. The training may comprise at least one optimization or tuning process, wherein a best parameter combination is determined. The training may be performed iteratively on the training data sets of different subjects. The processing unit 112 may be configured for considering different numbers of features for determining the analysis model by training the machine learning model with the training data set. The algorithm of the machine learning model may be applied to the training data set using a different number of features, e.g. depending on their ranking. The training may comprise n-fold cross validation to get a robust estimate of the model parameters. The training of the machine learning model may comprise at least one controlled learning process, wherein at least one hyper-parameter is chosen to control the training process. If necessary the training is step is repeated to test different combinations of hyper-parameters.

In particular subsequent to the training of the machine learning model, the processing unit 112 is configured for predicting the target variable on the test data set using the determined analysis model. The processing unit 112 may be configured for predicting the target variable for each subject based on the test data set of that subject using the determined analysis model. The processing unit 112 may be configured for predicting the target variable for each subject on the respective training and test data sets using the analysis model. The processing unit 112 may be configured for recording and/or storing both the predicted target variable per subject and the true value of the target variable per subject, for example, in at least one output file.

The processing unit 112 is configured for determining performance of the determined analysis model based on the predicted target variable and the true value of the target variable of the test data set. The performance may be characterized by deviations between predicted target variable and true value of the target variable. The machine learning system 110 may comprises at least one output interface 118. The output interface 118 may be designed identical to the communication interface 114 and/or may be formed integral with the communication interface 114. The output interface 118 may be configured for providing at least one output. The output may comprise at least one information about the performance of the determined analysis model. The information about the performance of the determined analysis model may comprises one or more of at least one scoring chart, at least one predictions plot, at least one correlations plot, and at least one residuals plot.

The model unit 116 may comprise a plurality of machine learning models, wherein the machine learning models are distinguished by their algorithm. For example, for building a regression model the model unit 116 may comprise the following algorithms k nearest neighbors (kNN), linear regression, partial last-squares (PLS), random forest (RF), and extremely randomized Trees (XT). For example, for building a classification model the model unit 116 may comprise the following algorithms k nearest neighbors (kNN), support vector machines (SVM), linear discriminant analysis (LDA), quadratic discriminant analysis (QDA), naïve Bayes (NB), random forest (RF), and extremely randomized Trees (XT). The processing unit 112 may be configured for determining an analysis model for each of the machine learning models by training the respective machine learning model with the training data set and for predicting the target variables on the test data set using the determined analysis models.

FIG. 2 shows an exemplary sequence of steps of a method according to the present invention. In step a), denoted with reference number 120, the input data is received via the communication interface 114. The method comprises pre-processing the input data, denoted with reference number 122. As outlined above, the pre-processing may comprise at least one filtering process for input data fulfilling at least one quality criterion. For example, the input data may be filtered to remove missing variables. For example, the pre-processing may comprise excluding data from subjects with less than a pre-defined minimum number of observations. In step b), denoted with reference number 124, the training data set and the test data set are determined by the processing unit 112. The method may further comprise at least one data aggregation and/or data transformation on both of the training data set and the test data set for each subject. The method may further comprise at least one feature extraction. The steps of data aggregation and/or data transformation and feature extraction are denoted with reference number 126 in FIG. 2. The feature extraction may comprise the ranking of features. In step c), denoted with reference number 128, the analysis model is determined by training a machine learning model comprising at least one algorithm with the training data set. In step d), denoted with reference number 130, the target variable is predicted on the test data set using the determined analysis model. In step e), denoted with reference number 132, performance of the determined analysis model is determined based on the predicted target variable and a true value of the target variable of the test data set

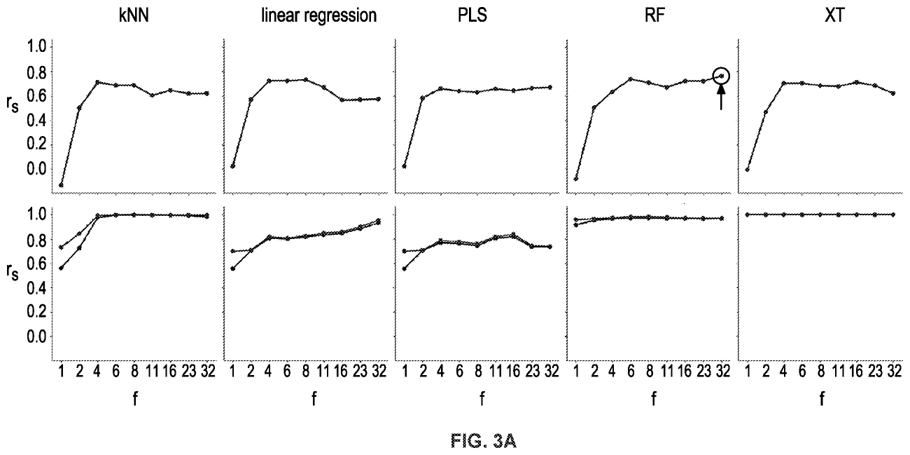

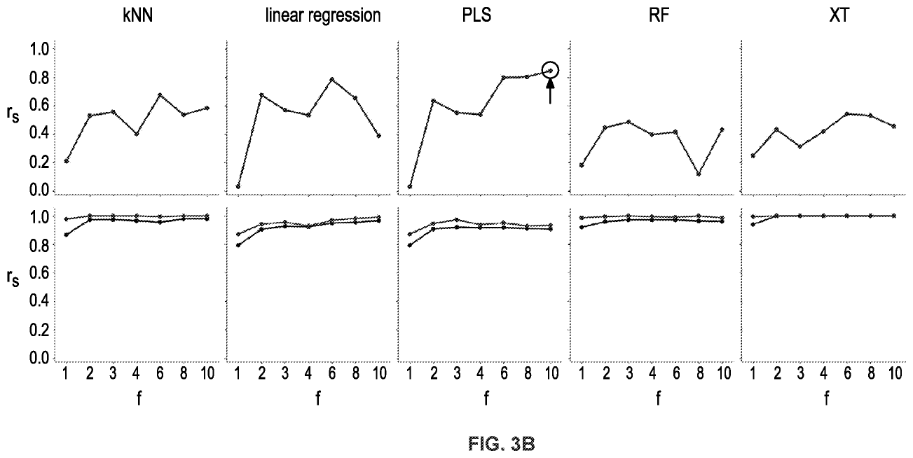

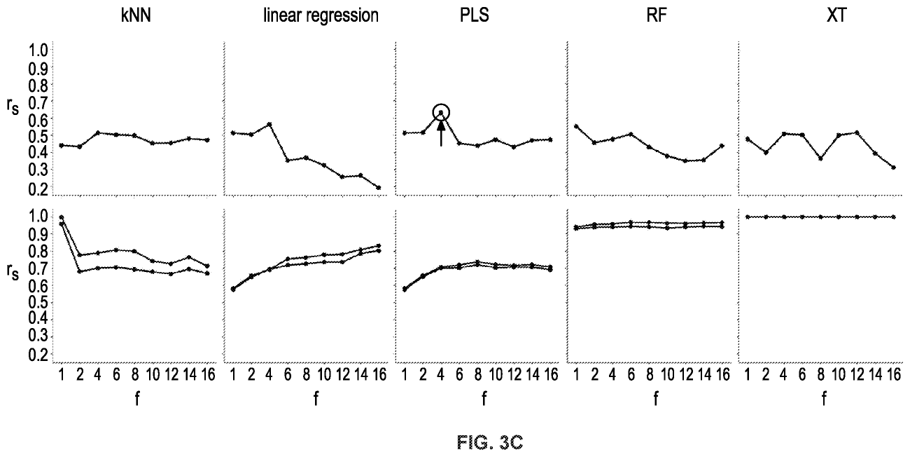

FIG. 3A, FIG. 3B, and FIG. 3C show embodiments of correlations plots for assessment of performance of an analysis model.

FIG. 3A show a correlations plot for analysis models, in particular regression models, for predicting an expanded disability status scale value indicative of multiple sclerosis. The input data was data from Floodlight POC study from 52 subjects.

In the prospective pilot study (FLOODLIGHT) the feasibility of conducting remote patient monitoring with the use of digital technology in patients with multiple sclerosis was evaluated. A study population was selected by using the following inclusion and exclusion criteria:

Key inclusion criteria:

Signed informed consent form

Able to comply with the study protocol, in the investigator's judgment

Age 18-55 years, inclusive

Have a definite diagnosis of MS, confirmed as per the revised McDonald 2010 criteria

EDSS score of 0.0 to 5.5, inclusive

Weight: 45-110 kg

For women of childbearing potential: Agreement to use an acceptable birth control method during the study period

Key exclusion criteria:

Severely ill and unstable patients as per investigator's discretion

Change in dosing regimen or switch of disease modifying therapy (DMT) in the last 12 weeks prior to enrollment

Pregnant or lactating, or intending to become pregnant during the study

It is a primary objective of this study to show adherence to smartphone and smartwatch-based assessments quantified as compliance level (%) and to obtain feedback from patients and healthy controls on the smartphone and smartwatch schedule of assessments and the impact on their daily activities using a satisfaction questionnaire. Furthermore, additional objectives are addressed, in particular, the association between assessments conducted using the Floodlight Test and conventional MS clinical outcomes was determined, it was established if Floodlight measures can be used as a marker for disease activity/progression and are associated with changes in MRI and clinical outcomes over time and it was determined if the Floodlight Test Battery can differentiate between patients with and without MS, and between phenotypes in patients with MS.

In addition to the active tests and passive monitoring, the following assessments were performed at each scheduled clinic visit:
Oral Version of SDMT
Fatigue Scale for Motor and Cognitive Functions (FSMC)
Timed 25-Foot Walk Test (T25-FW)
Berg Balance Scale (BBS)
9-Hole Peg Test (9HPT)
Patient Health Questionnaire (PHQ-9)
Patients with MS only:
Brain MRI (MSmetrix)
Expanded Disability Status Scale (EDSS)
Patient Determined Disease Steps (PDDS)
Pen and paper version of MSIS-29

While performing in-clinic tests, patients and healthy controls were asked to carry/wear smartphone and smartwatch to collect sensor data along with in-clinic measures. In summary, the results of the study showed that patients are highly engaged with the smartphone- and smartwatch-based assessments. Moreover, there is a correlation between tests and in-clinic clinical outcome measures recorded at baseline which suggests that the smartphone-based Floodlight Test Battery shall become a powerful tool to continuously monitor MS in a real-world scenario. Further, the smartphone-based measurement of turning speed while walking and performing U-turns appeared to correlate with EDSS.

For FIG. 3A, in total, 889 features from 7 tests were evaluated during model building using the method according to the present invention. The tests used for this prediction were the Symbol-Digits Modalities Test (SMDT) where the subject has to match as many symbols as possible to digits in a given time span; the pinching test, where the subject has to squeeze, using the thumb and index finger, as many tomatoes shown on the screen as possible in a given time span; the Draw-A-Shape test, where the subject has to trace shapes on the screen; the Standing Balance Test where the subject has to stand upright for 30 seconds; the 5 U-Turn test where the subject has to walk short spans followed by 180 degree turns; the 2 Minute Walking test, where the subject has to walk for two minutes; and finally the passive monitoring of the gait. The following table gives an overview of selected features used for prediction, test from which the feature was derived, short description of feature and ranking:

| feature | test | Description of feature | rank |
|---|---|---|---|
| logistic | Passive | Average per-step power coefficient | 1 |
| step_power_mean | Monitoring | (integral of variance in accelerometer | |
| (40-60 s) | | radius over per-step time span) for | |
| | | gait bouts spanning 40-60 s | |
| sigmoid turns_utt | U-TURN | Number of turns | 2 |
| log10 Gc_0_15 | SDMT | Mean Timegap between correct | 3 |
| | | responses from time 0 to 15 seconds | |
| sigmoid | U-TURN | maximum turn speed | 4 |
| turn_speed_max_utt | | | |
| logistic | 2MWT | Average per-step power coefficient | 5 |
| step_power_mean | | (integral of variance in accelerometer | |
| | | radius over per-step time span) | |
| sigmoid | U-TURN | minimum turn speed | 6 |
| turn_speed_min_utt | | | |
| sigmoid | Passive | Variance of per-step power coefficient | 7 |
| step_power_variance | Monitoring | for gait bouts spanning 60-90 s | |
| (60-90 s) | | | |
| logistic | Passive | Variance of per-step power coefficient | 8 |
| step_power_variance | Monitoring | for gait bouts spanning 40-60 s | |
| (40-60 s) | | | |
| sigmoid | Passive | Average per-step power coefficient | 9 |
| step_power_mean | Monitoring | (integral of variance in accelerometer | |
| (<20 s) | | radius over per-step time span) for | |
| | | gait bouts spanning <20 s | |
| span_duration_s_median_utt | U-TURN | median gait bout length | 10 |
| logistic | Passive | Variance of per-step power coefficient | 11 |
| step_power_variance | Monitoring | for gait bouts spanning 20-40 s | |
| (20-40 s) | | | |
| sigmoid | Passive | Variance of per-step power coefficient | 12 |
| step_power_variance | Monitoring | for gait bouts spanning 90-120 s | |
| (90-120 s) | | | |
| sigmoid | U-TURN | median turn speed | 13 |
| turn_speed_median_utt | | | |
| logistic | Passive | Average per-step power coefficient | 14 |
| step_power_mean | Monitoring | (integral of variance in accelerometer | |
| (60-90 s) | | radius over per-step time span) for | |
| | | gait bouts spanning 60-90 s | |
| sigmoid GcM_0_15 | SDMT | Maximal Timegap between correct | 15 |
| | | responses from time 0 to 15 seconds | |
| logistic | Passive | Average per-step power coefficient | 16 |
| step_power_mean | Monitoring | (integral of variance in accelerometer | |
| (20-40 s) | | radius over per-step time span) for | |
| | | gait bouts spanning 20-40 s | |
| logistic | Passive | Average per-step power coefficient | 17 |
| step_power_mean | Monitoring | (integral of variance in accelerometer | |
| (90-120 s) | | radius over per-step time span) for | |
| | | gait bouts spanning 90-120 s | |
| CCR_0_45 | SDMT | from time 0 to 45 seconds: Number of | 18 |
| | | correct responses within the longest | |
| | | sequence of overall consecutive | |
| | | correct responses | |
| span_duration_s_max_utt | U-TURN | maximum gait bout length | 19 |
| log10 R_Symbol_9 | SDMT | Number of total responses for symbol | 20 |
| | | 9: “.—” | |
| Gc_0_30 | SDMT | Mean Timegap between correct | 21 |
| | | responses from time 0 to 30 seconds | |
| sigmoid CCR_0_15 | SDMT | from time 0 to 15 seconds: Number of | 22 |
| | | correct responses within the longest | |
| | | sequence of overall consecutive | |
| | | correct responses | |
| sigmoid GM_0_15 | SDMT | Maximal Timegap between responses | 23 |
| | | from time 0 to 15 seconds | |
| sigmoid R_0_15 | SDMT | Number of total responses from time 0 | 24 |
| | | to 15 seconds | |
| log10 CR_Symbol_8 | SDMT | Number of correct responses for | 25 |
| | | symbol 8: “)” | |
| log10 CCR_0_30 | SDMT | from time 0 to 30 seconds: Number of | 26 |
| | | correct responses within the longest | |
| | | sequence of overall consecutive correct | |
| | | responses | |
| log10 G_0_15 | SDMT | Mean Timegap between responses | 27 |
| | | from time 0 to 15 seconds | |
| sigmoid CR_0_15 | SDMT | Number of correct responses from | 28 |
| | | time 0 to 15 seconds | |
| log10 Gc_0_45 | SDMT | Mean Timegap between correct | 29 |
| | | responses from time 0 to 45 seconds | |
| log10 R_Symbol_8 | SDMT | Number of total responses for symbol | 30 |
| | | 8: “)” | |
| log10 R_0_30 | SDMT | Number of total responses from time 0 | 31 |
| | | to 30 seconds | |
| sigmoid CR_0_30 | SDMT | Number of correct responses from | 32 |
| | | time 0 to 30 seconds | |

FIG. 3A shows the Spearman correlation coefficient rₛ between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. The curves in the lower row show results for “all” and “Mean” obtained from predicting the target variable on the training data. “Mean” refers to the prediction on the average value of all observations per subject. “all” refers to the prediction on all individual observations. For assessing the performance of any machine learning model, the results from the test data (top row) were considered more reliable. It was found that the best performing regression model is RF with 32 features included in the model, having an rₛ value of 0.77, indicated with circle and arrow.

The following gives more detailed description of the tests. The tests are typically computer-implemented on a data acquisition device such as a mobile device as specified elsewhere herein.

(1) Tests for Passive Monitoring of Gait and Posture: Passive Monitoring

The mobile device is, typically, adapted for performing or acquiring data from passive monitoring of all or a subset of activities In particular, the passive monitoring shall encompass monitoring one or more activities performed during a predefined window, such as one or more days or one or more weeks, selected from the group consisting of: measurements of gait, the amount of movement in daily routines in general, the types of movement in daily routines, general mobility in daily living and changes in moving behavior.

Typical passive monitoring performance parameters of interest:
a. frequency and/or velocity of walking;
b. amount, ability and/or velocity to stand up/sit down, stand still and balance
c. number of visited locations as an indicator of general mobility;
d. types of locations visited as an indicator of moving behavior.

(2) Test for Cognitive Capabilities: SMDT (Also Denoted as eSDMT)

The mobile device is also, typically, adapted for performing or acquiring a data from an computer-implemented Symbol Digit Modalities Test (eSDMT). The conventional paper SDMT version of the test consists of a sequence of 120 symbols to be displayed in a maximum 90 seconds and a reference key legend (3 versions are available) with 9 symbols in a given order and their respective matching digits from 1 to 9. The smartphone-based eSDMT is meant to be self-administered by patients and will use a sequence of symbols, typically, the same sequence of 110 symbols, and a random alternation (form one test to the next) between reference key legends, typically, the 3 reference key legends, of the paper/oral version of SDMT. The eSDMT similarly to the paper/oral version measures the speed (number of correct paired responses) to pair abstract symbols with specific digits in a predetermined time window, such as 90 seconds time. The test is, typically, performed weekly but could alternatively be performed at higher (e.g. daily) or lower (e.g. bi-weekly) frequency. The test could also alternatively encompass more than 110 symbols and more and/or evolutionary versions of reference key legends. The symbol sequence could also be administered randomly or according to any other modified pre-specified sequence.

Typical eSDMT performance parameters of interest:
1. Number of correct responses
   a. Total number of overall correct responses (CR) in 90 seconds (similar to oral/paper SDMT)
   b. Number of correct responses from time 0 to 30 seconds (CR₀₋₃₀)
   c. Number of correct responses from time 30 to 60 seconds (CR₃₀₋₆₀)
   d. Number of correct responses from time 60 to 90 seconds (CR₆₀₋₉₀)
   e. Number of correct responses from time 0 to 45 seconds (CR₀₋₄₅)
   f Number of correct responses from time 45 to 90 seconds (CR₄₅₋₉₀)
   g. Number of correct responses from time i to j seconds (CRᵢ₋ⱼ), where i, j are between 1 and 90 seconds and i<j.
2. Number of errors
   a. Total number of errors (E) in 90 seconds
   b. Number of errors from time 0 to 30 seconds (E₀₋₃₀)
   c. Number of errors from time 30 to 60 seconds (E₃₀₋₆₀)
   d. Number of errors from time 60 to 90 seconds (E₆₀₋₉₀)
   e. Number of errors from time 0 to 45 seconds (E₀₋₄₅)
   f Number of errors from time 45 to 90 seconds (E₄₅₋₉₀)
   g. Number of errors from time i to j seconds (Eᵢ₋ⱼ), where i,j are between 1 and 90 seconds and i<j.
3. Number of responses
   a. Total number of overall responses (R) in 90 seconds
   b. Number of responses from time 0 to 30 seconds (R₀₋₃₀)
   c. Number of responses from time 30 to 60 seconds (R₃₀₋₆₀)
   d. Number of responses from time 60 to 90 seconds (R₆₀₋₉₀)
   e. Number of responses from time 0 to 45 seconds (R₀₋₄₅)
   f Number of responses from time 45 to 90 seconds (R₄₅₋₉₀)
4. Accuracy rate
   a. Mean accuracy rate (AR) over 90 seconds: AR=CR/R
   b. Mean accuracy rate (AR) from time 0 to 30 seconds: AR₀₋₃₀=CR₀₋₃₀/R₀₋₃₀
   c. Mean accuracy rate (AR) from time 30 to 60 seconds: AR₃₀₋₆₀=CR₃₀₋₆₀/R₃₀₋₆₀
   d. Mean accuracy rate (AR) from time 60 to 90 seconds: AR₆₀₋₉₀=CR₆₀₋₉₀/R₆₀₋₉₀
   e. Mean accuracy rate (AR) from time 0 to 45 seconds: AR₀₋₄₅=CR₀₋₄₅/R₀₋₄₅
   f. Mean accuracy rate (AR) from time 45 to 90 seconds: AR₄₅₋₉₀=CR₄₅₋₉₀/R₄₅₋₉₀
5. End of task fatigability indices
   a. Speed Fatigability Index (SFI) in last 30 seconds: SFI₆₀₋₉₀=CR₆₀₋₉₀/max (CR₀₋₃₀, CR₃₀₋₆₀)
   b. SFI in last 45 seconds: SFI₄₅₋₉₀=CR₄₅₋₉₀/CR₀₋₄₅
   c. Accuracy Fatigability Index (AFI) in last 30 seconds: AFI₆₀₋₉₀=AR₆₀₋₉₀/max (AR₀₋₃₀, AR₃₀₋₆₀)
   d. AFI in last 45 seconds: AFI₄₅₋₉₀=AR₄₅₋₉₀/AR₀₋₄₅
6. Longest sequence of consecutive correct responses
   a. Number of correct responses within the longest sequence of overall consecutive correct responses (CCR) in 90 seconds
   b. Number of correct responses within the longest sequence of consecutive correct responses from time 0 to 30 seconds (CCR₀₋₃₀)
   c. Number of correct responses within the longest sequence of consecutive correct responses from time 30 to 60 seconds (CCR₃₀₋₆₀)
   d. Number of correct responses within the longest sequence of consecutive correct responses from time 60 to 90 seconds (CCR₆₀₋₉₀)
   e. Number of correct responses within the longest sequence of consecutive correct responses from time 0 to 45 seconds (CCR₀₋₄₅)
   f. Number of correct responses within the longest sequence of consecutive correct responses from time 45 to 90 seconds (CCR₄₅₋₉₀)
7. Time gap between responses
   a. Continuous variable analysis of gap (G) time between two successive responses
   b. Maximal gap (GM) time elapsed between two successive responses over 90 seconds
   c. Maximal gap time elapsed between two successive responses from time 0 to 30 seconds (GM₀₋₃₀)
   d. Maximal gap time elapsed between two successive responses from time 30 to 60 seconds (GM₃₀₋₆₀)
   e. Maximal gap time elapsed between two successive responses from time 60 to 90 seconds (GM₆₀₋₉₀)
   f. Maximal gap time elapsed between two successive responses from time 0 to 45 seconds (GM₀₋₄₅)
   g. Maximal gap time elapsed between two successive responses from time 45 to 90 seconds (GM₄₅₋₉₀)
8. Time Gap between correct responses
   a. Continuous variable analysis of gap (Gc) time between two successive correct responses
   b. Maximal gap time elapsed between two successive correct responses (GcM) over 90 seconds
   c. Maximal gap time elapsed between two successive correct responses from time 0 to 30 seconds (GcM₀₋₃₀)
   d. Maximal gap time elapsed between two successive correct responses from time 30 to 60 seconds (GcM₃₀₋₆₀)
   e. Maximal gap time elapsed between two successive correct responses from time 60 to 90 seconds (GcM₆₀₋₉₀)
   f. Maximal gap time elapsed between two successive correct responses from time 0 to 45 seconds (GcM₀₋₄₅)
   g. Maximal gap time elapsed between two successive correct responses from time 45 to 90 seconds (GcM₄₅₋₉₀)
9. Fine finger motor skill function parameters captured during eSDMT
   a. Continuous variable analysis of duration of touchscreen contacts (Tts), deviation between touchscreen contacts (Dts) and center of closest target digit key, and mistyped touchscreen contacts (Mts) (i.e contacts not triggering key hit or triggering key hit but associated with secondary sliding on screen), while typing responses over 90 seconds
   b. Respective variables by epochs from time 0 to 30 seconds: Tts₀₋₃₀, Dts₀₋₃₀, MtS₀₋₃₀
   c. Respective variables by epochs from time 30 to 60 seconds: Tts₃₀₋₆₀, Dts₃₀₋₆₀, MtS₃₀₋₆₀
   d. Respective variables by epochs from time 60 to 90 seconds: Tts₆₀₋₉₀, Dts₆₀₋₉₀, Mts₆₀₋₉₀
   e. Respective variables by epochs from time 0 to 45 seconds: Tts₀₋₄₅, Dts₀₋₄₅, Mts₀₋₄₅
   f. Respective variables by epochs from time 45 to 90 seconds: Tts₄₅₋₉₀, Dts₄₅₋₉₀, Mts₄₅₋₉₀
10. Symbol-specific analysis of performances by single symbol or cluster of symbols
   a. CR for each of the 9 symbols individually and all their possible clustered combinations
   b. AR for each of the 9 symbols individually and all their possible clustered combinations
   c. Gap time (G) from prior response to recorded responses for each of the 9 symbols individually and all their possible clustered combinations
   d. Pattern analysis to recognize preferential incorrect responses by exploring the type of mistaken substitutions for the 9 symbols individually and the 9 digit responses individually.
11. Learning and cognitive reserve analysis
   a. Change from baseline (baseline defined as the mean performance from the first 2 administrations of the test) in CR (overall and symbol-specific as described in #9) between successive administrations of eSDMT
   b. Change from baseline (baseline defined as the mean performance from the first 2 administrations of the test) in AR (overall and symbol-specific as described in #9) between successive administrations of eSDMT
   c. Change from baseline (baseline defined as the mean performance from the first 2 administrations of the test) in mean G and GM (overall and symbol-specific as described in #9) between successive administrations of eSDMT
   d. Change from baseline (baseline defined as the mean performance from the first 2 administrations of the test) in mean Gc and GcM (overall and symbol-specific as described in #9) between successive administrations of eSDMT
   e. Change from baseline (baseline defined as the mean performance from the first 2 administrations of the test) in SFI₆₀₋₉₀ and SFI₄₅₋₉₀ between successive administrations of eSDMT
   f. Change from baseline (baseline defined as the mean performance from the first 2 administrations of the test) in AFI₆₀₋₉₀ and AFI₄₅₋₉₀ between successive administrations of eSDMT
   g. Change from baseline (baseline defined as the mean performance from the first 2 administrations of the test) in Tts between successive administrations of eSDMT
   h. Change from baseline (baseline defined as the mean performance from the first 2 administrations of the test) in Dts between successive administrations of eSDMT
   i. Change from baseline (baseline defined as the mean performance from the first 2 administrations of the test) in Mts between successive administrations of eSDMT.

(3) Tests for Active Gait and Posture Capabilities: U-Turn Test (Also Denoted as Five U-Turn Test, 5UTT) and 2MWT

A sensor-based (e.g. accelerometer, gyroscope, magnetometer, global positioning system [GPS]) and computer implemented test for measures of ambulation performances and gait and stride dynamics, in particular, the 2-Minute Walking Test (2MWT) and the Five U-Turn Test (5UTT).

In one embodiment, the mobile device is adapted to perform or acquire data from the TwoMinute Walking Test (2MWT). The aim of this test is to assess difficulties, fatigability or unusual patterns in long-distance walking by capturing gait features in a two-minute walk test (2MWT). Data will be captured from the mobile device. A decrease of stride and step length, increase in stride duration, increase in step duration and asymmetry and less periodic strides and steps may be observed in case of disability progression or emerging relapse. Arm swing dynamic while walking will also be assessed via the mobile device. The subject will be instructed to “walk as fast and as long as you can for 2 minutes but walk safely”. The 2MWT is a simple test that is required to be performed indoor or outdoor, on an even ground in a place where patients have identified they could walk straight for as far as ≥200 meters without U-turns. Subjects are allowed to wear regular footwear and an assistive device and/or orthotic as needed. The test is typically performed daily.

Typical 2MWT performance parameters of particular interest:
1. Surrogate of walking speed and spasticity:
   a. Total number of steps detected in, e.g., 2 minutes (ΣS)
   b. Total number of rest stops if any detected in 2 minutes (ΣRs)
   c. Continuous variable analysis of walking step time (WsT) duration throughout the 2MWT
   d. Continuous variable analysis of walking step velocity (WsV) throughout the 2MWT (step/second)
   e. Step asymmetry rate throughout the 2MWT (mean difference of step duration between one step to the next divided by mean step duration): SAR=meanΔ(WsTₓ−WsTₓ₊₁)/(120/ΣS)
   f. Total number of steps detected for each epoch of 20 seconds (ΣS_{t, t+20})
   g. Mean walking step time duration in each epoch of 20 seconds: WsTt_{, t+20}=20/ΣS_{t, t+20}
   h. Mean walking step velocity in each epoch of 20 seconds: WsV_{t, t+20}=ΣS_{t, t+20}/20
   i. Step asymmetry rate in each epoch of 20 seconds: SAR_{t, t+20}=meanΔ_{t, t+20}(WsTₓ−WsTₓ₊₁)/(20/ΣS_{t, t+20})
   j. Step length and total distance walked through biomechanical modelling
2. Walking fatigability indices:
   a. Deceleration index: DI=WsV₁₀₀₋₁₂₀/max (WsV₀₋₂₀, WsV₂₀₋₄₀, WsV₄₀₋₆₀)
   b. Asymmetry index: AI=SAR₁₀₀₋₁₂₀/min (SAR₀₋₂₀, SAR₂₀₋₄₀, SAR₄₀₋₆₀)

In another embodiment, the mobile device is adapted to perform or acquire data from the Five U-Turn Test (5UTT). The aim of this test is to assess difficulties or unusual patterns in performing U-turns while walking on a short distance at comfortable pace. The 5UTT is required to be performed indoor or outdoor, on an even ground where patients are instructed to “walk safely and perform five successive U-turns going back and forward between two points a few meters apart”. Gait feature data (change in step counts, step duration and asymmetry during U-turns, U-turn duration, turning speed and change in arm swing during U-turns) during this task will be captured by the mobile device. Subjects are allowed to wear regular footwear and an assistive device and/or orthotic as needed. The test is typically performed daily.

Typical 5UTT performance parameters of interest:
1. Mean number of steps needed from start to end of complete U-turn (ΣSu)
2. Mean time needed from start to end of complete U-turn (Tu)
3. Mean walking step duration: Tsu=Tu/ΣSu
4. Turn direction (left/right)
5. Turning speed (degrees/sec)

FIG. 3B show a correlations plot for analysis models, in particular regression models, for predicting a forced vital capacity (FVC) value indicative of spinal muscular atrophy. The input data was data from OLEOS study from 14 subjects. In total, 1326 features from 9 tests were evaluated during model building using the method according to the present invention. The following table gives an overview of selected features used for prediction, test from which the feature was derived, short description of feature and ranking:

| Performance parameter | test | description | rank |
|---|---|---|---|
| lmax_pressure_min | Distal Motor | The minimum value of each | 1 |
| | Function test | maximum pressure reading | |
| | (Tap-The- | per finger tap | |
| | Monster) | | |
| log10 DTA_F | Squeeze-A- | the mean lag time between | 2 |
| | Shape | first and second fingers | |
| | | touch the screen of failed | |
| | | pinches | |
| log10 | Voice test | Mean absolute difference | 3 |
| norm_pct_diff_Mean_MFCCs_9 | | of successive cycles of the | |
| | | 9^{th} Mel Frequency Cepstral | |
| | | Coefficient (MFCC) | |
| log10 std_Mean_MFCCs_8 | Voice test | The standard deviation of | 4 |
| | | the mean value of successive | |
| | | cycles of the 8th | |
| | | MFCC | |
| logistic fatigue_index | Voice test | An estimate for vocal | 5 |
| | | fatigue defined as the ratio of | |
| | | max duration of the first | |
| | | half to max duration of the | |
| | | second half | |
| log10 DTA_S | Squeeze-A- | the mean lag time between | 6 |
| | Shape | first and second fingers | |
| | | touch the screen of successful | |
| | | pinches | |
| sigmoid LINE_TOP_TO_BOTTOM_errSQRT | Draw-A- | square root of the drawing | 7 |
| | Shape | error for the line top-to-bottom | |
| | | shape | |
| log10 DTA_0_15 | Squeeze-A- | the mean lag time between | 8 |
| | Shape | first and second fingers | |
| | | touch the screen between | |
| | | time window 0 s-15 s | |
| log10 DTA_15_30 | Squeeze-A- | the mean lag time between | 9 |
| | Shape | first and second fingers | |
| | | touch the screen between | |
| | | time window 15 s-30 s | |
| log10 DTA | Squeeze-A- | DTA = mean(pinch_start − | 10 |
| | Shape | finger_down): the mean lag | |
| | | time between first and second | |
| | | fingers touch the screen | |

FIG. 3B shows the Spearman correlation coefficient rₛ between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. The curves in the lower row show results for “all” and “Mean” obtained from predicting the target variable on the training data. “Mean” refers to the prediction on the average value of all observations per subject. “all” refers to the prediction on all individual observations. For assessing the performance of any machine learning model, the results from the test data (top row) were considered more reliable. It was found that the best performing regression model is PLS with 10 features included in the model, having an rₛ value of 0.8, indicated with circle and arrow.

The following gives more detailed description of the tests. The tests are typically computer-implemented on a data acquisition device such as a mobile device as specified elsewhere herein.

(1) Tests for Central Motor Functions: Draw a Shape Test and Squeeze a Shape Test

The mobile device may be further adapted for performing or acquiring a data from a further test for distal motor function (so-called “draw a shape test”) configured to measure dexterity and distal weakness of the fingers. The dataset acquired from such test allow identifying the precision of finger movements, pressure profile and speed profile.

The aim of the “Draw a Shape” test is to assess fine finger control and stroke sequencing. The test is considered to cover the following aspects of impaired hand motor function: tremor and spasticity and impaired hand-eye coordination. The patients are instructed to hold the mobile device in the untested hand and draw on a touchscreen of the mobile device 6 prewritten alternating shapes of increasing complexity (linear, rectangular, circular, sinusoidal, and spiral; vide infra) with the second finger of the tested hand “as fast and as accurately as possible” within a maximum time of for instance 30 seconds. To draw a shape successfully the patient's finger has to slide continuously on the touchscreen and connect indicated start and end points passing through all indicated check points and keeping within the boundaries of the writing path as much as possible. The patient has maximum two attempts to successfully complete each of the 6 shapes. Test will be alternatingly performed with right and left hand. User will be instructed on daily alternation. The two linear shapes have each a specific number “a” of checkpoints to connect, i.e “a-1” segments. The square shape has a specific number “b” of checkpoints to connect, i.e. “b-1” segments. The circular shape has a specific number “c” of checkpoints to connect, i.e. “c-1” segments. The eight-shape has a specific number “d” of checkpoints to connect, i.e “d-1” segments. The spiral shape has a specific number “e” of checkpoints to connect, “e-1” segments. Completing the 6 shapes then implies to draw successfully a total of “(2a+b+c+d+e−6)” segments.

Typical Draw a Shape test performance parameters of interest:

Based on shape complexity, the linear and square shapes can be associated with a weighting factor (Wf) of 1, circular and sinusoidal shapes a weighting factor of 2, and the spiral shape a weighting factor of 3. A shape which is successfully completed on the second attempt can be associated with a weighting factor of 0.5. These weighting factors are numerical examples which can be changed in the context of the present invention.
1. Shape completion performance scores:
   a. Number of successfully completed shapes (0 to 6) (ΣSh) per test
   b. Number of shapes successfully completed at first attempt (0 to 6) (ΣSh₁)
   c. Number of shapes successfully completed at second attempt (0 to 6) (ΣSh₂)
   d. Number of failed/uncompleted shapes on all attempts (0 to 12) (ΣF)
   e. Shape completion score reflecting the number of successfully completed shapes adjusted with weighting factors for different complexity levels for respective shapes (0 to 10) (Σ[Sh*Wf])
   f. Shape completion score reflecting the number of successfully completed shapes adjusted with weighting factors for different complexity levels for respective shapes and accounting for success at first vs second attempts (0 to 10) (Σ[Sh₁*Wf]+Σ[Sh₂*Wf*0.5])
   g. Shape completion scores as defined in #1e, and #1f may account for speed at test completion if being multiplied by 30/t, where t would represent the time in seconds to complete the test.
   h. Overall and first attempt completion rate for each 6 individual shapes based on multiple testing within a certain period of time: (ΣSh₁)/(ΣSh₁+ΣSh₂+ΣF) and (ΣSh₁+ΣSh₂)/(ΣSh₁+ΣSh₂+ΣF).
2. Segment completion and celerity performance scores/measures:
   (analysis based on best of two attempts [highest number of completed segments] for each shape, if applicable)
      a. Number of successfully completed segments (0 to [2a+b+c+d+e−6]) (ΣSe) per test
      b. Mean celerity ([C], segments/second) of successfully completed segments: C=ΣSe/t, where t would represent the time in seconds to complete the test (max 30 seconds)
      c. Segment completion score reflecting the number of successfully completed segments adjusted with weighting factors for different complexity levels for respective shapes (Σ[Se*Wf])
      d. Speed-adjusted and weighted segment completion score (Σ[Se*Wf]*30/t), where t would represent the time in seconds to complete the test.
      e. Shape-specific number of successfully completed segments for linear and square shapes (ΣSe_{LS})
      f. Shape-specific number of successfully completed segments for circular and sinusoidal shapes (ΣSe_{CS})
      g. Shape-specific number of successfully completed segments for spiral shape (ΣSe_{S})
      h. Shape-specific mean linear celerity for successfully completed segments performed in linear and square shape testing: C_{L}=ΣSe_{LS}/t, where t would represent the cumulative epoch time in seconds elapsed from starting to finishing points of the corresponding successfully completed segments within these specific shapes.
      i. Shape-specific mean circular celerity for successfully completed segments performed in circular and sinusoidal shape testing: C_{C}=ΣSe_{CS}/t, where t would represent the cumulative epoch time in seconds elapsed from starting to finishing points of the corresponding successfully completed segments within these specific shapes.
      j. Shape-specific mean spiral celerity for successfully completed segments performed in the spiral shape testing: C_{S}=ΣSe_{S}/t, where t would represent the cumulative epoch time in seconds elapsed from starting to finishing points of the corresponding successfully completed segments within this specific shape.
3. Drawing precision performance scores/measures:
   (analysis based on best of two attempts[highest number of completed segments] for each shape, if applicable)
      a. Deviation (Dev) calculated as the sum of overall area under the curve (AUC) measures of integrated surface deviations between the drawn trajectory and the target drawing path from starting to ending checkpoints that were reached for each specific shapes divided by the total cumulative length of the corresponding target path within these shapes (from starting to ending checkpoints that were reached).
      b. Linear deviation (Dev_{L}) calculated as Dev in #3a but specifically from the linear and square shape testing results.
      c. Circular deviation (Dev_{C}) calculated as Dev in #3a but specifically from the circular and sinusoidal shape testing results.
      d. Spiral deviation (Dev_{S}) calculated as Dev in #3a but specifically from the spiral shape testing results.
      e. Shape-specific deviation (Dev₁₋₆) calculated as Dev in #3a but from each of the 6 distinct shape testing results separately, only applicable for those shapes where at least 3 segments were successfully completed within the best attempt.
      f. Continuous variable analysis of any other methods of calculating shape-specific or shape-agnostic overall deviation from the target trajectory.
4.) Pressure profile measurement
   i) Exerted average pressure
   ii) Deviation (Dev) calculated as the standard deviation of pressure

The distal motor function (so-called “squeeze a shape test”) may measure dexterity and distal weakness of the fingers. The dataset acquired from such test allow identifying the precision and speed of finger movements and related pressure profiles. The test may require calibration with respect to the movement precision ability of the subject first.

The aim of the Squeeze a Shape test is to assess fine distal motor manipulation (gripping & grasping) & control by evaluating accuracy of pinch closed finger movement. The test is considered to cover the following aspects of impaired hand motor function: impaired gripping/grasping function, muscle weakness, and impaired hand-eye coordination. The patients are instructed to hold the mobile device in the untested hand and by touching the screen with two fingers from the same hand (thumb+second or thumb+third finger preferred) to squeeze/pinch as many round shapes (i.e. tomatoes) as they can during 30 seconds. Impaired fine motor manipulation will affect the performance. Test will be alternatingly performed with right and left hand. User will be instructed on daily alternation.

Typical Squeeze a Shape test performance parameters of interest:
1. Number of squeezed shapes
   a. Total number of tomato shapes squeezed in 30 seconds (ΣSh)
   b. Total number of tomatoes squeezed at first attempt (ΣSh₁) in 30 seconds (a first attempt is detected as the first double contact on screen following a successful squeezing if not the very first attempt of the test)
2. Pinching precision measures:
   a. Pinching success rate (PsR) defined as ΣSh divided by the total number of pinching (ΣP) attempts (measured as the total number of separately detected double finger contacts on screen) within the total duration of the test.
   b. Double touching asynchrony (DTA) measured as the lag time between first and second fingers touch the screen for all double contacts detected.
   c. Pinching target precision (P_{TP}) measured as the distance from equidistant point between the starting touch points of the two fingers at double contact to the centre of the tomato shape, for all double contacts detected.
   d. Pinching finger movement asymmetry (P_{FMA}) measured as the ratio between respective distances slid by the two fingers (shortest/longest) from the double contact starting points until reaching pinch gap, for all double contacts successfully pinching.
   e. Pinching finger velocity (P_{FS}) measured as the speed (mm/sec) of each one and/or both fingers sliding on the screen from time of double contact until reaching pinch gap, for all double contacts successfully pinching.
   f. Pinching finger asynchrony (PFA) measured as the ratio between velocities of respective individual fingers sliding on the screen (slowest/fastest) from the time of double contact until reaching pinch gap, for all double contacts successfully pinching.
   g. Continuous variable analysis of 2a to 2f over time as well as their analysis by epochs of variable duration (5-15 seconds)
   h. Continuous variable analysis of integrated measures of deviation from target drawn trajectory for all tested shapes (in particular the spiral and square)
3.) Pressure profile measurement
i) Exerted average pressure
ii) Deviation (Dev) calculated as the standard deviation of pressure

More typically, the Squeeze a Shape test and the Draw a Shape test are performed in accordance with the method of the present invention. Even more specifically, the performance parameters listed in the Table 1 below are determined.

The data acquisition device may be further adapted for performing or acquiring a data from a further test for central motor function (so-called “voice test”) configured to measure proximal central motoric functions by measuring voicing capabilities.

(2) Cheer-the-Monster Test, Voice Test:

The term “Cheer-the-Monster test”, as used herein, relates to a test for sustained phonation, which is, in an embodiment, a surrogate test for respiratory function assessments to address abdominal and thoracic impairments, in an embodiment including voice pitch variation as an indicator of muscular fatigue, central hypotonia and/or ventilation problems. In an embodiment, Cheer-the-Monster measures the participant's ability to sustain a controlled vocalization of an “aaah” sound. The test uses an appropriate sensor to capture the participant's phonation, in an embodiment a voice recorder, such as a microphone.

In an embodiment, the task to be performed by the subject is as follows: Cheer the Monster requires the participant to control the speed at which the monster runs towards his goal. The monster is trying to run as far as possible in 30 seconds. Subjects are asked to make as loud an “aaah” sound as they can, for as long as possible. The volume of the sound is determined and used to modulate the character's running speed. The game duration is 30 seconds so multiple “aaah” sounds may be used to complete the game if necessary.

(3) Tap-the-Monster Test:

The term “Tap the Monster test”, as used herein, relates to a test designed for the assessment of distal motor function in accordance with MFM D3 (Bérard C et al. (2005), Neuromuscular Disorders 15:463). In an embodiment, the tests are specifically anchored to MFM tests 17 (pick up ten coins), 18 (go around the edge of a CD with a finger), 19 (pick up a pencil and draw loops) and 22 (place finger on the drawings), which evaluate dexterity, distal weakness/strength, and power. The game measures the participant's dexterity and movement speed. In an embodiment, the task to be performed by the subject is as follows: Subject taps to on monsters appearing randomly at 7 different screen positions.

FIG. 3C show a correlations plot for analysis models, in particular regression models, for predicting a total motor score (TMS) value indicative of Huntington's disease. The input data was data from HD OLE study, ISIS 44319-CS2 from 46 subjects. The ISIS 443139-CS2 study is an Open Label Extension (OLE) for patients who participated in Study ISIS 443139-CS1. Study ISIS 443139-CS1 was a multiple-ascending dose (MAD) study in 46 patients with early manifest HD aged 25-65 years, inclusive. In total, 43 features were evaluated from one test, the Draw-A-Shape test (see above), were evaluated during model building using the method according to the present invention. The following table gives an overview of selected features used for prediction, test from which the feature was derived, short description of feature and ranking:

| Performance parameter | test | description | rank |
|---|---|---|---|
| log10 SPIRAL_sp_cov | Draw-A- | The coefficient of variation | 1 |
| | Shape | in the drawing velocity | |
| | | of the Spiral shape | |
| SPIRAL_hausD | Draw-A- | The maximum hausdorff | 2 |
| | Shape | distance between drawn | |
| | | and reference shape - as a | |
| | | proxy for maximumm | |
| | | drawing error for the Spiral | |
| | | shape | |
| log10 SQUARE_acc_celerity | Draw-A- | The number of way- | 3 |
| | Shape | points hit (accuracy) | |
| | | divided by the time take to | |
| | | complete the Square | |
| | | shape | |
| sigmoid SQUARE_Mag_areaError | Draw-A- | | 4 |
| | Shape | | |

FIG. 3C shows the Spearman correlation coefficient rₛ between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. The curves in the lower row show results for “all” and “Mean” in the lower row are results obtained from predicting the target variable on the training data. “Mean” refers to the prediction on the average value of all observations per subject. “all” refers to the prediction on all individual observations. For assessing the performance of any machine learning model, the results from the test data (top row) were considered more reliable. It was found that the best performing regression model is PLS with 4 features included in the model, having an rₛ value of 0.65, indicated with circle and arrow.

### LIST OF REFERENCE NUMBERS

110 machine learning system
112 processing unit
114 communication interface
116 model unit
118 output interface
120 step a)
122 pre-processing
124 step b)
126 transformation and feature extraction
128 step c)
130 step d)
132 step e)

1. A machine learning system (110) for determining at least one analysis model for predicting at least one target variable indicative of a disease status comprising:
at least one communication interface (114) configured for receiving input data, wherein the input data comprises a set of historical digital biomarker feature data, wherein the set of historical digital biomarker feature data comprises a plurality of measured values indicative of the disease status to be predicted, wherein the historical digital biomarker feature data is experimental data determined by at least one mobile device which comprises a plurality of different measurement values per subject relating to symptoms of the disease, wherein the input data is determined in an active test using the mobile device such as at least one cognition test and/or at least one hand motor function test and/or or at least one mobility test;
at least one model unit (116) comprising at least one machine learning model comprising at least one algorithm;
at least one processing unit (112), wherein the processing unit (112) is configured for determining at least one training data set and at least one test data set from the input data set, wherein the processing unit (112) is configured for determining the analysis model by training the machine learning model with the training data set, wherein the training is a process of determining parameters of the algorithm of machine learning model on the training data set, wherein the training is performed iteratively on the training data sets of different subjects, wherein the analysis model is a regression model, wherein the algorithm of the machine learning model is at least one algorithm selected from the group consisting of: k nearest neighbors (kNN); linear regression;
partial last-squares (PLS); random forest (RF); and extremely randomized Trees (XT), or wherein the analysis model is a classification model, wherein the algorithm of the machine learning model is at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT), wherein the processing unit (112) is configured for predicting the target variable on the test data set using the determined analysis model, wherein the processing unit (112) is configured for determining performance of the determined analysis model based on the predicted target variable and a true value of the target variable of the test data set,
wherein the machine learning system (110) comprises at least one output interface (118), wherein the output interface (118) is configured for providing at least one output, wherein the output comprises at least one information about the performance of the determined analysis model, wherein the information about the performance of the determined analysis model comprises one or more of at least one scoring chart, at least one predictions plot, at least one correlations plot, and at least one residuals plot,
wherein the model unit (116) comprises a plurality of machine learning models,
wherein the machine learning models are distinguished by their algorithm, wherein the processing unit (112) is configured for determining an analysis model for each of the machine learning models by training the respective machine learning model with the training data set and for predicting the target variables on the test data set using the determined analysis models, wherein the processing unit (112) is configured for determining performance of each of the determined analysis models based on the predicted target variables and the true value of the target variable of the test data set, wherein the processing unit (112) is configured for determining the analysis model having the best performance.

2. The machine learning system (110) of claim 1, wherein the disease whose status is to be predicted is multiple sclerosis and the target variable is an expanded disability status scale (EDSS) value, or wherein the disease whose status is to be predicted is spinal muscular atrophy and the target variable is a forced vital capacity (FVC) value, or wherein the disease whose status is to be predicted is Huntington's disease and the target variable is a total motor score (TMS) value.

3. The machine learning system (110) of claim 1, wherein the processing unit (112) is configured for generating and/or creating per subject of the input data a training data set and a test data set, wherein the test data set comprises data of one subject, wherein the training data set comprises the other input data.

4. The machine learning system (110) of claim 1, wherein the processing unit (112) is configured for extracting features from the input data, wherein the processing unit (112) is configured for ranking the features by using a maximum-relevance-minimum-redundancy technique.

5. The machine learning system (110) of claim 4, wherein the processing unit (112) is configured for considering different numbers of features for determining the analysis model by training the machine learning model with the training data set.

6. The machine learning system (110) of claim 1, wherein the processing unit (112) is configured for pre-processing the input data, wherein the pre-processing comprises at least one filtering process for input data fulfilling at least one quality criterion.

7. The machine learning system (110) of claim 1, wherein the processing unit (112) is configured for performing one or more of at least one stabilizing transformation; at least one aggregation; and at least one normalization for the training data set and for the test data set.

8. A computer-implemented method for determining at least one analysis model for predicting at least one target variable indicative of a disease status, using the machine learning system (110) of claim 1, wherein the method comprises the following steps:
a) receiving input data via at least one communication interface (114), wherein the input data comprises a set of historical digital biomarker feature data, wherein the set historical digital biomarker feature data comprises a plurality of measured values indicative of the disease status to be predicted;
at least one processing unit (112):
b) determining at least one training data set and at least one test data set from the input data set;
c) determining the analysis model by training a machine learning model comprising at least one algorithm with the training data set;
d) predicting the target variable on the test data set using the determined analysis model;
e) determining performance of the determined analysis model based on the predicted target variable and a true value of the target variable of the test data set.

9. The method of claim 8, wherein in step c) a plurality of analysis models is determined by training a plurality of machine learning models with the training data set, wherein the machine learning models are distinguished by their algorithm, wherein in step d) a plurality of target variables is predicted on the test data set using the determined analysis models, wherein in step e) the performance of each of the determined analysis models is determined based on the predicted target variables and the true value of the target variable of the test data set, wherein the method further comprises determining the analysis model having the best performance.

10. Computer program for determining at least one analysis model for predicting at least one target variable indicative of a disease status, configured for causing a computer or computer network to fully or partially perform the method for determining at least one analysis model for predicting at least one target variable indicative of a disease status as in the method of claim 8, when executed on the computer or computer network, wherein the computer program is configured to perform at least steps b) to e) of the method for determining at least one analysis model for predicting at least one target variable indicative of a disease status according to any one of the preceding claims referring to a method.

11. The machine learning system (110) of claim 1 wherein the machine learning system is for determining an analysis model for predicting one or more of an expanded disability status scale (EDSS) value indicative of multiple sclerosis, a forced vital capacity (FVC) value indicative of spinal muscular atrophy, or a total motor score (TMS) value indicative of Huntington's disease.
